(19) United States
(12) Patent Application Publication
(10) Pub. No.: US 2007/0179161 A1
(43) Pub. Date: Aug. 2, 2007

(54) Pyrazolopyrimidine compounds and their use in medicine

(75) Inventors: Martin Parratt, Cambridge (GB); Justin Fairfield Bower, Cambridge (GB); Douglas Williamson, Cambridge (GB); Andrew Cansfield, Cambridge (GB)

Correspondence Address: BANNER & WITCOFF, LTD., 1100 13th STREET, N.W., SUITE 1200, WASHINGTON, DC, 20005-4051, US

(73) Assignee: VERNALIS (CAMBRIDGE) LIMITED., Abington (GB)

(21) Appl. No.: 10/551,177

(22) PCT Filed: Mar. 18, 2004
(86) PCT No.: PCT/GB04/01214

§ 371 Date: Dec. 6, 2006

(30) Foreign Application Priority Data
Mar. 31, 2003 (GB) 0307389.7
May 29, 2003 (GB) 0312296.7
Aug. 13, 2003 (GB) 0319028.7
Nov. 5, 2003 (GB) 0325854.8

(51) Int. Cl. A61K 31/519; C07D 487/02

(52) U.S. Cl. 514/259.3; 544/280

(57) ABSTRACT

Compounds of formula (I) or salts, N-oxides, hydrates or solvates thereof are inhibitors of kinase activity, and useful for the treatment of, for example, cancer, psoriasis or restenosis: wherein ring A is an optionally substituted carbocyclic or heterocyclic radical. Alk represents an optionally substituted divalent C1-C₆ alkylene radical. n is 0 or 1. Q represents a radical of formula -(Alk¹)ₚ (X)ᵣ-(Alk²)ₛ-Z wherein in any compatible combination Z is hydrogen or an optionally substituted carbocyclic or heterocyclic ring; Alk¹ and Alk² are optionally substituted divalent C₁-C₆ alkylene radicals which may contain a —O—, —S— or —NR^{A}— link, wherein R^{A} is hydrogen or C₁-C₆ alkyl; X represents —O—, —S—, —(C═O)—, —(C═S)—, —SO₂—, —SO—, —C(═O)O—, —OC(═O)—, —C(═O)NR^{A—, —NR} ^{A}C(═O)—, —C(═S)NR^{A}, —NR^{A}C(═S)—, —SO₂NR^{A}—, —NR^{A}SO₂—, —OC(═O)NR^{A—, —NR}^{A}C(═O)O—, or —NR^{A}— wherein R^{A} is hydrogen or C₁-C₆ alkyl. p, r and s are independently 0 or 1. R₁ represents a radical -(Alk³)ₐ-(Y)b-(Alk⁴)_{d}-B wherein a, b and d are independently 0 or 1; Alk³ and Alk⁴ are optionally substituted divalent C,-C3 alkylene radicals; Y represents a monocyclic divalent carbocyclic or heterocyclic radical having from 5 to 8 ring atoms, —O—, —S—, or —NR^{A}— wherein R^{A} is hydrogen or C₁-C₆ alkyl; B represents hydrogen or halo, or an optionally substituted monocyclic carbocyclic or heterocyclic ring having from 5 to 8 ring atoms, or in the case where Y is —NR^{A}— and b is 1, then R^{A} and the radical -(Alk⁴)_{d}-B taken together with the nitrogen to which they are attached may form an optionally substituted heterocyclic ring. R represents hydrogen, halo, C₁-C₆ alkyl, C1-C₆ alkoxy, C₁-C₆ alkylthio, phenyl, benzyl, cycloalkyl with 3 to 6 ring atoms, or a monocyclic heterocyclic group having 5 or 6 ring atoms.

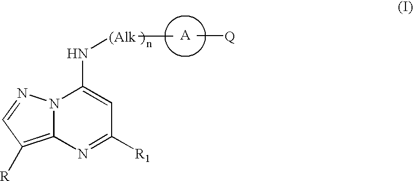

This invention relates to the use of a class of substituted amino pyrazolo[1,5-a]pyrimidines in relation to diseases which are mediated by excessive or inappropriate kinase activity, for example CDK2 and/or PDK1 and/or CHK1 activity, such as cancers.

### BACKGROUND TO THE INVENTION

CDK2

Uncontrolled cell proliferation is a hallmark of cancer. Tumor cells typically have damage to genes which play a part in regulation of the cell division cycle. Cyclin-dependent kinases (CDKs) play critical roles in regulating the transitions between different phases of the cell cycle. The serine/threonine kinase CDK2 is essential for normal cell cycling and plays a key role in disorders arising form aberrant cell cycling. Inhibitors of CDK2 are therefore useful for the treatment of various types of cancer and other conditions related to abnormal cell proliferation. Flavopyridol (M. D. Losiewiecz et al., Biochem. Biophys. Res. Commun., 1994, 201, 589-595), which is currently in clinical trials, displays modest selectivity for inhibition of CDKs over other kinases but inhibits CDK1, CDK2, and CDK4 with equal potency. A purine based derivative, roscovitine (CYC-202) (W. F. De Azevedo et al., Eur. J. Biochem., 1997, 243, 518-526), similarly displays selectivity for CDKs over other kinases and is also in clinical trials.

PDK1

For a normal cell to acquire the phenotype of a malignant tumour cell, several barriers must be overcome. One of the most important is the ability to evade programmed cell death (apoptosis). Mutations downregulating various aspects of the cell-death machinery are therefore a hallmark of cancer. The PI-3 kinase-AKT pathway transmits survival signals from growth factor receptors to downstream effectors. In a substantial number of tumour cells, this pathway is inappropriately activated by either amplification of the PI-3 kinase or Akt genes, or loss of expression of the PTEN tumour suppressor. Activation of this pathway enables cancer cells to survive under conditions where normal cells would die, enabling the continued expansion of the tumour. The 3′-phosphoinositide-dependent protein kinase-1 (PDK1) is an essential component of the PI-3 kinase-AKT pathway. In the presence of PIP3, the second messenger generated by PI-3 kinase, PDK1 phosphorylates Akt on threonine 308, a modification essential for Akt activation. PDK1 also phosphorylates the corresponding threonine residues of certain other pro-survival kinases including SGK and p70 S6 kinase (Vanhaesebroeck B & Alessi D R. Biochem J 346, 561-576 (2000)). Experiments with genetically modified mice indicate that reducing PDK1 activity to 10% of the normal level is surprisingly well tolerated (Lawlor M A et al. EMBO J 21, 3728-3738 (2002)). Certain cancer cells, however, appear to be less able to tolerate antisense-mediated reductions in PDK1 activity (Flynn P et al. Curr Biol. 10, 1439-1442 (2000)). Moreover, both celecoxib and UCN-01, small molecules that inhibit PDK1 both in vitro and in cells, are capable of inducing apoptosis in cultured tumour cells (Arico et al. J. Biol. Chem. 277, 27613-27621 (2002);Sato et al. Oncogene 21, 1727-1738 (2002)). Agents that inhibit the PDK1 kinase may therefore be useful for the therapy of cancer.

CHK1

Many standard cancer chemotherapeutic agents act primarily through their ability to induce DNA damage causing tumour growth inhibition. However, these agents cause cell cycle arrest by induction of checkpoints at either S-phase or G2-M boundary. The G2 arrest allows the cell time to repair the damaged DNA before entering mitosis. Chk1 and an unrelated serine/threonine kinase, Chk2, play a central role in arresting the cell cycle at the G2-M boundary (O'Connell et al EMBO J (1997) vol 16 p545-554). Chk1/2 induce this checkpoint by phosphorylating serine 216 of the CDC25 phosphatase, inhibiting the removal of two inactivating phosphates on cyclin dependent kinases (CDKs) (Zheng et al Nature (1998) vol 395 p507-510). Another overlapping pathway mediated by p53 also elicits cycle arrest in response to DNA-damage. However, p53 is mutationally inactivated in many cancers, resulting in a partial deficiency in their ability to initiate a DNA-repair response. If Chk1 activity is also inhibited in p53-negative cancers, all ability to arrest and repair DNA in response to DNA-damage is removed resulting in mitotic catastrophe and enhancing the effect of the DNA damaging agents (Konarias et al Oncogene (2001) vol 20 p7453-7463; Bunch and Eastman Clin. Can. Res. (1996) vol 2 p791-797; Tenzer and Pruschy Curr. Med Chem (2003) vol 3 p35-46). In contrast, normal cells would be relatively unaffected due to retention of a competent p53-mediated cell-cycle arrest pathway. A Chk1 inhibitor (UCN-01) is now in phase I clinical trials for improving the efficacy of current DNA-damage inducing chemotherapeutic regimens (Sausville et al, J. Clinical Oncology (2001) vol19 p2319-2333).

### BRIEF DESCRIPTION OF THE INVENTION

The present invention relates to the use of a class of amino pyrazolo[1,5-a]pyrimidine compounds as kinase inhibitors, for example CDK2 and/or PDK1 and/or CHK1 inhibitors, for example for inhibition of cancer cell proliferation. A core 7-amino pyrazolo[1,5-a]pyrimidine ring with aromatic substitution on the amino group are principle characterising features of the compounds with which the invention is concerned.

### DETAILED DESCRIPTION OF THE INVENTION

According to the present invention there is provided the use of a compound of formula (I) or a salt, N-oxide, hydrate or solvate thereof, in the preparation of a composition for inhibition of kinase activity: 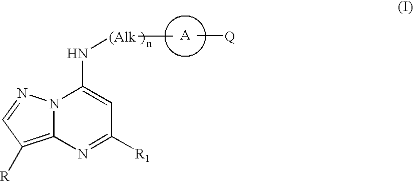
wherein
Ring A is an optionally substituted carbocyclic or heterocyclic radical,
Alk represents an optionally substituted divalent C₁-C₆ alkylene radical;
n is 0 or 1;
Q represents a radical of formula -(Alk¹)ₚ-(X)ᵣ-(Alk²)ₛ-Z wherein in any compatible combination
   Z is hydrogen or an optionally substituted carbocyclic or heterocyclic ring,
   Alk¹ and Alk² are optionally substituted divalent C₁-C₆ alkylene radicals which may contain a —O—, —S—or —NR^{A}— link, wherein R^{A} is hydrogen or C₁-C₆ alkyl,
   X represents —O—, —S—, —(C═O)—, —(C═S)—, —SO₂—, —SO—, —C(═O)O—, —OC(═O)—, —C(═O)NR^{A}—, —NR^{A}C(═O)—, —C(═S)NR^{A}—, —NR^{A}C(═S)—, —SO₂NR^{A}, —NR^{A}SO₂—, —OC(═O)NR^{A}—, —NR^{A}C(═O)O—, or —NR^{A}— wherein R^{A} is hydrogen or C₁-C₆ alkyl,
   p, r and s are independently 0 or 1, and
R₁ represents a radical -(Alk³)ₐ-(Y)_{b}-(Alk⁴)_{d}-B wherein
   a, b and d are independently 0 or 1,
   Alk³ and Alk⁴ are optionally substituted divalent C₁-C₃ alkylene radicals,
   Y represents a monocyclic divalent carbocyclic or heterocyclic radical having from 5 to 8 ring atoms, —O—, —S—, or —NR^{A}— wherein R^{A} is hydrogen or C₁-C₆ alkyl,
   B represents hydrogen or halo, or an optionally substituted monocyclic carbocyclic or heterocyclic ring having from 5 to 8 ring atoms, or in the case where Y is —NR^{A}— and b is 1, then R^{A} and the radical -(Alk⁴)_{d}-B taken together with the nitrogen to which they are attached may form an optionally substituted heterocyclic ring,
R represents hydrogen, halo, C₁-C₆ alkyl, C₁-C₆ alkoxy, C₁-C₆ alkylthio, phenyl, benzyl, cycloalkyl with 3 to 6 ring atoms, or a monocyclic heterocyclic group having 5 or 6 ring atoms.

In particular, the invention relates to the use of such compounds in the preparation of a composition for inhibiting CDK2 and/or PDK1 and/or CHK1 activity.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers means a saturated hydrocarbon chain having from a to b carbon atoms and two unsatisfied valences.

As used herein the unqualified term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “aryl” refers to a mono-, bi- or tri-cyclic carbocyclic aromatic radical, and to two such radicals covalently linked to each other, Illustrative of such radicals are phenyl, biphenyl and napthyl.

As used herein the unqualified term “carbocyclic” refers to a cyclic radical whose ring atoms are all carbon and to two such cyclic radicals covalently linked to each other, and includes aryl, and cycloalkyl radicals. Typically, carbocyclic radicals will have from 3 to 14 ring atoms.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Typically, a heterocyclic radical will have from 5 to 14 ring atoms. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with at least one substituent, for example selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro and chloro), trifluoromethyl, trifluoromethoxy, nitro, nitrile (—CN), oxo, phenyl, phenoxy, benzyl, benzyloxy, monocyclic carbocyclic or heterocyclic having from 5 to 7 ring atoms, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHSO₂R^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl group or phenyl. The term “optional substituent” includes one of the foregoing substituent groups.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

Some compounds of the invention contain one or more actual or potential chiral centres because of the presence of asymmetric carbon atoms. The presence of several asymmetric carbon atoms gives rise to a number of diastereoisomers with R or S stereochemistry at each chiral centre. The invention includes all such diastereoisomers and mixtures thereof.

The Ring A

Ring A is an optionally substituted carbocyclic or heterocyclic radical, preferably monocyclic aryl or heteroaryl radical. Examples of ring A include phenyl, naphthyl, 2-, 3- and 4-pyridyl, 5-pyrimidinyl, 2- and 3-thienyl, 2- and 3-furyl, piperazinyl, pyrrolidinyl, and thiazolinyl. Currently it is preferred that ring A is a phenyl ring.

Ring A may be optionally substituted by any of the substituents listed above in the definition of “optionally substituted”. Examples of optional substiuents on ring A or ring B include methyl, ethyl, methylenedioxy, ethylenedioxy, methoxy, ethoxy, methylthio, ethylthio, hydroxy, hydroxymethyl, hydroxyethyl, mercapto, mercaptomethyl, mercaptoethyl, amino, mono- and di-methylamino, mono- and di-ethylamino, fluoro, chloro, bromo, cyano, N-morpholino, N-piperidinyl, N-piperazinyl (the latter being optionally C₁-C₆ alkyl- or benzyl-substituted on the free ring nitrogen), dimethylaminosulfonyl, phenylsulfonyl or phenoxy.

The Radical -(Alk)ₙ-

When present, the Alk radical acts as a spacer radical between the amino group on the pyrazolo[1,5-a]pyrimidine ring and the ring A, and may be, for example —CH₂—, —CH₂CH₂—, —CH₂CH(CH₃)—, —CH₂CH₂CH₂—, —CH═CH—, —CH₂CH═CH—, —CH₂CH═CHCH₂—, —CH═CHCH═CH—, —C≡C—, —CH₂C≡C—, or —CH₂C≡CCH₂—. Presently it is preferred that Alk, when present, is —CH₂— or —CH₂CH₂—.

However, in another preferred class of compounds with which the invention is concerned, n may be 0 so that the ring A is directly linked to the amino group on the pyrazolo[1,5-a]pyrimidine ring.

The Q Substituent

In the simplest structures with which the invention is concerned, each of p, r and s may be 0, and Z may be hydrogen, so that ring A is simply a carbocyclic or heterocyclic radical, preferably monocyclic aryl or heteroaryl radical, optionally substituted as discussed above. Substituents which are presently preferred, when ring A is optionally substituted phenyl, are dimethylaminosulfonyl, phenylsulfonyl or phenoxy especially in the 4-position.

In other simple structures, p, r and s may again each be 0, and Z may be an optionally substituted carbocyclic or heterocyclic ring, for example phenyl, cyclopentyl, cyclohexyl, pyridyl, morpholino, piperidinyl, or piperazyl ring. In such cases, Z is a direct substituent in the optionally substituted ring A.

In more complex structures with which the invention is concerned, one or more of p, r and s may be 1, and Z may be hydrogen or an optionally substituted carbocyclic or heterocyclic ring. For example, p and/or s may be 1 and r may be 0, so that Z is linked to ring A by an alkylene radical, for example a C₁-C₃ alkylene radical, which is optionally substituted. In other cases each of p, r, and s may be 1, in which cases, Z is linked to ring A by an alkylene radical which is interrupted by the hetero atom-containing X radical. In still other cases, p and s may be 0 and r may be 1, in which case Z is linked to ring A via the hetero atom-containing X radical. In a preferred example of the latter case, ring A is phenyl, p and s are each 0, X is —SO₂— or —O— on the 4-position of the phenyl ring A, and Z is phenyl (optionally substituted).

In other preferred embodiments, p is 0, r is 1, and he is a sulfonamide radical —NR^{A}SO₂— or a carboxamide radical —NR^{A}C(═O)— (R^{A} being as defined above, but preferably hydrogen), with the N atom linked to the ring A. In such cases s may be 1 and Z may be hydrogen, so that the group Q is an alkylsulfonamido or carboxamido substituent in the ring A; or s may be 0 and Q may be an optionally substituted carbocyclic or heterocyclic ring such as optionally substituted phenyl, eg 4-methylphenyl, so that the group Q is an optionally substituted phenylsulfonamido or carboxamido substituent in the ring A.

In another preferred subclass of compounds of the invention, p is 0, r is 1, and X is a sulfonamide radical —NR^{A}SO₂— (R^{A} being as defined above), with the S atom linked to the ring, ie a compound of structure (IA):

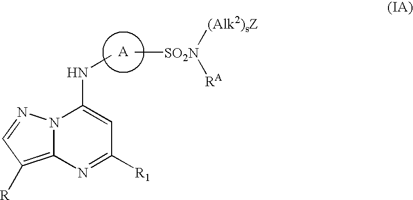

In compounds of structure (IA) R^{A} may be, for example methyl or phenyl, and -Alk²)ₛZ may be, for example methyl or hydrogen; or R^{A} and -Alk²)ₛZ, taken together with the nitrogen to which they are attached may form a ring such as:

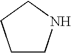

In a further preferred subclass of compounds of the invention, p is 0, r is 1, and X is a sulfonyl radical —SO₂— ie a compound of structure (IB): 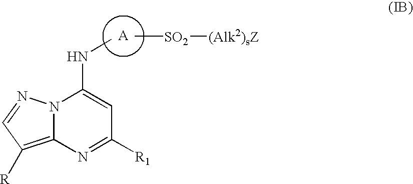
The Substituent R₁

R₁ represents a radical -(Alk³)ₐ-(Y)_{b}-(Alk⁴)_{d}-B as defined above.

In one class of compounds of the invention a, b and d are all 0, and B is hydrogen or halo, so that the pyrimidine ring is either unsubstituted or substituted by halogen, for example chloro or bromo.

In another class of compounds of the invention, B is an optionally substituted monocyclic carbocyclic or heterocyclic ring, for example cyclopentyl, cyclohexyl, phenyl, 2-, 3-, or 4-pyridyl, 2-, or 3-thienyl, 2-, or 3-furanyl, pyrrolyl, pyranyl, or piperidinyl ring. Of the foregoing, cyclohexyl, and piperidin-1-yl are presently preferred. Optional substituents in ring B may be any of the substituents listed above in the definition of “optionally substituted”. Examples of optional substituents on ring B include methyl, ethyl, methoxy, ethoxy, methylenedioxy, ethylenedioxy, methylthio, ethylthio, hydroxy, hydroxymethyl, hydroxyethyl, mercapto, mercaptomethyl, mercaptoethyl, amino, mono- and di-methylamino, mono- and di-ethylamino, fluoro, chloro, bromo, cyano, N-morpholino, N-piperidinyl, N-piperazinyl (the latter being optionally C₁-C₈ alkyl- or benzyl-substituted on the free ring nitrogen). Of the foregoing substituents, amino, is currently preferred, particularly when in the 4-position of a cyclohexyl or piperidin-1-yl ring B. In such cases, ring B is linked to the pyrimidine ring via linker radical of various types depending on the values of a, b and d, and the identities of Alk³, Y and Alk⁴. For example, when b is 0, the ring B is linked to the pyrimidine ring via an optionally substituted C₁-C₆ alkylene radical, methylene being presently preferred; and when a and d are 0 and b is 1 the ring B is linked to the pyrimidine ring via an oxygen or sulfur link or via an amino link —NR^{A}— wherein R^{A} is hydrogen or C₁-C₆ alkyl such as methyl or ethyl. In the latter case, ie where a and d are each 0 and b is 1, it is presently preferred that Y is —O— or —NH—,

In another class of compounds of the invention b is 0, at least one of a and d is 1, and B is hydrogen, so that the pyrimidine ring is substituted by a C₁-C₆ alkyl group, for example methyl, ethyl, and n- or iso-propyl, which may itself be substituted by substituents listed above in the definition of “optionally substituted. Examples of optional substituents include methoxy, ethoxy, methylthio, ethylthio, hydroxy, hydroxymethyl, hydroxyethyl, mercapto, mercaptomethyl, mercaptoethyl, amino, mono- and di-methylamino, mono- and di-ethylamino, fluoro, chloro, bromo, and cyano.

In a further class of compounds of the invention a is 1 or 0, b is 1, Y is —NR^{A}—, and the radical -(Alk⁴)_{d}-B taken together with R_{A} and the nitrogen to which they are attached form an optionally substituted heterocyclic ring such as a ring piperidinyl, morpholinyl or piperazinyl ring, optionally substituted, for example, by hydroxy, mercapto, methoxy, ethoxy, methylthio, ethylthio, amino, mono- or dimethyl amino, mono- or diethyl amino, nitro, or cyano. In the case of a piperazinyl ring, the second ring nitrogen may optionally be substituted by, for example methyl or ethyl.

Specific examples of R₁ include those present in the compounds of the Examples herein, especially cyclohexyloxy; cyclohexylamino; cyclohexylmethyl, and piperidin-1-ylmethyl, all optionally substituted in the ring by amino, particularly in the 4-position, for example by amino, or hydroxy.

The Group R

R may be, for example, hydrogen, chloro, bromo methyl, ethyl, n-propyl, iso-propyl, n-, sec- or tert-butyl, methoxy, methylthio, ethoxy, ethylthio, phenyl, benzyl, cyclopropyl, cyclopentyl, cyclohexyl, 2-, 3-, or 4-pyridyl, phenyl, pyridyl, morpholino, piperidinyl, or piperazyl ring. At present it is preferred that R be chloro, bromo, cyclopentyl, cyclopropyl or isopropyl.

Specific compounds with which the invention is concerned include those identified in the Examples.

Novel compounds of formula (I) as discussed also form an aspect of the invention, particularly those wherein n is 0, ring A is optionally substituted phenyl (for example 3-chlorophenyl or 3-methoxyphenyl), Q is dimethylaminosulfonyl, phenylsulfonyl or phenoxy, R¹ is 4-aminocyclohexyloxy; 4-aminocyclohexylamino; 4-hydroxycyclohexylamino, 4-aminocyclohexylmethyl, or 4-aminopiperidin-1-ylmethyl, and R is chloro, bromo, cyclopentyl, cyclopropyl or isopropyl.

Compounds with which the invention is concerned may be prepared by literature methods, such as those of the preparative Examples herein, and methods analogous thereto.

For example, compounds of the invention wherein R₁ is hydrogen or halo may be prepared by reacting the chloro or dichloro compound (II) with the amine (III), 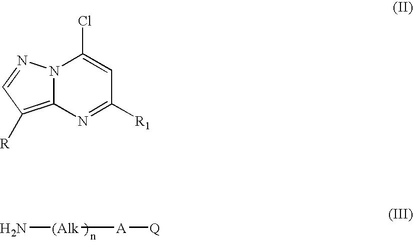
and in the case where R₁ is halo, separating the desired compound (I) from any resultant contaminant regioisomer (IV):

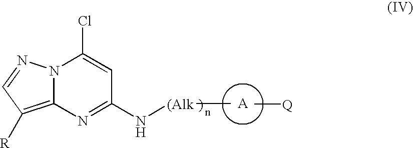

To prepared compounds of the invention wherein R₁ is a radical —(Y)ₐ—B the general synthetic procedure is based on the coupling of compounds (V) and (VI) 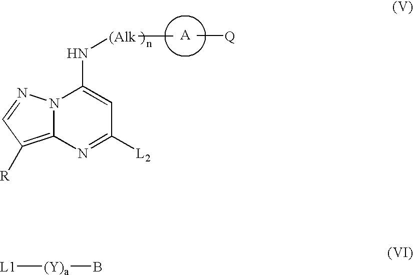
wherein L1 and L2 represent components of a leaving group L1L2.

Thus, to prepare compounds (I) wherein R₁ is —(Y)ₐ—B wherein a=0 and B is an aryl or heteroaryl ring, a compound of formula (VII) wherein Z is an N-protecting group may be reacted with the corresponding aryl or heteroaryl borohydrate compound (VIII) to prepare an intermediate compound (IX), from which the N-protecting group Z¹ may be removed to prepare the desired compound (I).

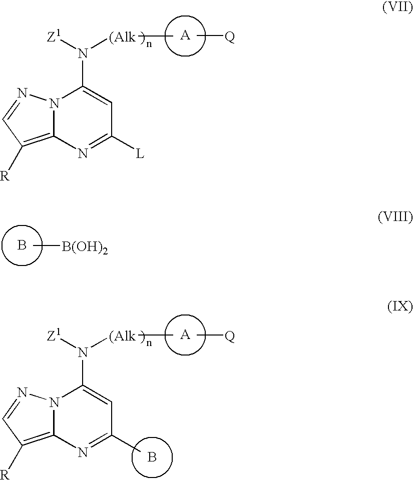

The starting compound (II) may be prepared by reaction of a compound (V) with an amine (VI):

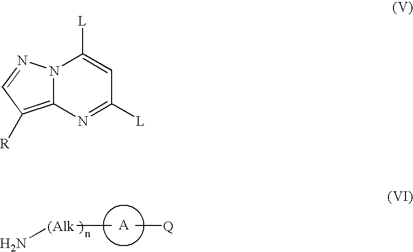

In the above formulae (II)-(VI), L signifies a leaving group such as halo, for example chloro. Ring A, Alk, Q and n are as defined in relation to formula (I).

Likewise, to prepare compounds (I) wherein R₁ is —(Y)ₐ—B wherein a=1, and Y is —O— the compound (VII), where L is chloro, for example, may be reacted with the hydroxy compound HY—B.

The compounds of the invention are inhibitors of kinases, for example CDK2 and/or PDK1 and/or CHK1, and are thus useful in the treatment of diseases which are mediated by excessive or inappropriate activity of such kinases, such as cancers, leukemias and other disease states associated with uncontrolled cell proliferation such as psoriasis and restenosis

Accordingly, the invention also provides:
(i) a method of treatment of diseases or conditions mediated by excessive or inappropriate kinase activity, for example CDK2 and/or PDK1 and/or CHK1 activity in mammals, particularly humans, which method comprises administering to the mammal an amount of a compound of formula (I) as defined above, or a salt, hydrate or solvate thereof, effective to inhibit said kinase activity.; and
(ii) a compound of formula (I) as defined above, or a salt hydrate or solvate thereof, for use in human or veterinary medicine, particularly in the treatment of diseases or conditions mediated by excessive or inappropriate kinase activity, for example CDK2 and/or PDK1 and/or CHK1 activity;

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The following non-limiting Examples illustrate the invention:

In the Examples, reactions that are specified as being carried out in a microwave oven were conducted in a Smith Synthesizer. Proton NMR experiments were conducted on a Bruker DPX400 ultra shield NMR spectrometer in the solvent specified.

LC-MS: Method A

| HPLC: | | HP1100 | | |
|---|---|---|---|---|
| Column: | | Luna 3 μm, C18(2), 30 mm × 4.6 mm i.d. | | |
| | | from Phenomenex | | |
| Temperature: | | 22° C. | | |
| Solvents: | | A - Water + 10 mmol ammonium acetate + 0.08% | | |
| | | (v/v) formic acid | | |
| | | B - 95% Acetonitrile / 5% Solvent A + 0.08% | | |
| | | (v/v) formic acid | | |
| Flow rate: | | 2 ml/min | | |
| | Time (mins) | % Solvent A | % Solvent B | Flow (ml/min) |
| | 0 | 95 | 5 | 2 |
| | 0.25 | 95 | 5 | 2 |
| | 2.50 | 5 | 95 | 2 |
| | 2.55 | 5 | 95 | 3 |
| | 3.60 | 5 | 95 | 3 |
| | 3.65 | 5 | 95 | 3 |
| | 3.70 | 5 | 95 | 2 |
| | 3.75 | 95 | 5 | 2 |

| Gradient | |
|---|---|
| Total acquisition time is 3.75 minutes | |
| Detection: | UV detection at 230 nm, 254 nm and 270 nm |
| Mass Spec: | HP1100 MSD, Series A |
| | Ionisation is positive or negative ion electrospray |
| | Molecular weight scan range is 120-1000 |

### EXAMPLE 1

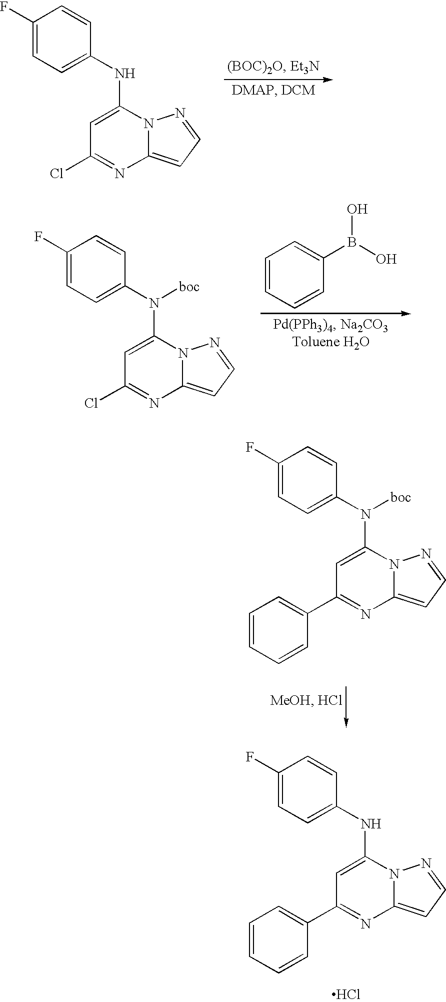

### Step 1

### 5-Chloro-7-(4-fluorophenylamino)pyrazolo[1,5-a]pyrimidine

To a solution of 5,7-dichloropyrazolo[1,5-a]pyrimidine¹ (0.35 g, 1.86 mmol) in ethanol (15 cm³) was added 4-fluoroaniline (0.35 cm³, 3.72 mmol). The reaction mixture was heated under reflux for 1 hour. The reaction mixture was concentrated in vacuo and the product purified on silica eluting with 15% ethyl acetate in hexanes, to yield the title compound as a white solid (0.42 g, 86%).
1. T. Novinson et al., Journal of Medicinal Chemistry (1976), 19(4), 512-16.

δ_{H} (400 MHz; d₄-MeOH) 8.02 (1H, d, J 2.2 Hz), 7.40-7.36 (2H, m), 7.21 (2H, t, J 6.7), 6.32 (1H, d, J 2.2 Hz), 5.97 (1H, s). m/z 263 and 265 (each M+H, 100% and 30%) retention time 2.54 min (Method A).

### Step 2

### 5-Chloro-7-(N-tert-butoxycarbonyl-4-fluorophenylamino)pyrazolo[1,5-a]pyrimidine

To a solution of 5-chloro-7-(4-fluorophenylamino)pyrazolo[1,5-a]pyrimidine (0.15 g, 0.57 mmol) in dichloromethane (10 cm³) was added di-tert-butyl dicarbonate (0.37 g, 1.71 mmol), triethylamine (0.096 cm³, 0.69 mmol) and 4-dimethylaminopyridine (0.01 g, 0.082 mmol). The reaction mixture was stirred at room temperature for 16 h. The reaction was diluted with water (30 cm³) and extracted with dichloromethane (3×20 cm³). The combined organic fractions were washed with brine then dried with magnesium sulphate and concentrated in vacuo. The product was purified on silica eluting with 20% ethyl acetate in hexanes, to yield the title compound as a white solid (0.191 g, 92%).

δ_{H} (400 MHz; d-CHCl₃) 8.09 (1H, d, J 2.3 Hz), 7.29-7.25 (2H, m), 6.99 (2H, t, J 8.1), 6.63 (1H, d, J 2.3 Hz), 6.60 (1H, s), 1.30 (9H, s).

### Step 3

### 5-Phenyl-7-(N-tert-butoxycarbonyl-4-fluorophenylamino)pyrazolo[1,5-a]pyrimidine

To a solution of 5-chloro-7-(N-tert-butoxycarbonyl-4-fluorophenylamino)pyrazolo[1,5-a]pyrimidine (0.05 g, 0.14 mmol) in toluene (3.5 cm³) and water (1 cm³) was added phenyl boronic acid (0.02 g, 0.16 mmol) and sodium carbonate (0.031 g, 0.29 mmol). The solution was degassed by bubbling nitrogen through the reaction mixture for 5 min. Tetrakis(triphenylphosphine)palladium(0) (0.015 g, 0.012 mmol) was added to the mixture and the reaction was heated at reflux for 16 h. The reaction mixture was concentrated in vacuo and purified on silica eluting with 20% ethyl acetate in hexanes to yield the title compound as an off-white solid (0.048 g, 86%).

δ_{H} (400 MHz; d-CHCl₃) 8.11 (1H, d, J 2.3 Hz), 7.99-7.97 (2H, m), 7.44-7.42 (3H, m), 7.34-7.31 (2H, m), 7.08 (1H, s), 6.97 (2H, t, J 8.3 Hz), 6.73 (1H, d, J 2.3 Hz), 1.31 (9H, s). m/z 405 (M+H, 80%), 349 (M+H-56, 70%), 305 (M+H-100, 100%), retention time 2.92 min (Method A).

### Step 4

### 5-Phenyl-7-(4-fluorophenylamino)pyrazolo[1,5-a]pyrimidine hydrochloride

To a solution of 5-phenyl-7-(N-tert-butoxycarbonyl-4-fluorophenylamino)pyrazolo[1,5-a]pyrimidine (0.045 g, 0.11 mmol) in methanol (1 cm³) was added a solution of hydrochloric acid (3 M in methanol, 10 cm³). The reaction mixture was stirred at room temperature for 3 h then concentrated in vacuo. The product was purified by crystalisation from ethyl acetate, to yield the title compound as a white solid (0.016 g, 42%).

δ_{H} (400 MHz; d₄-MeOH) 8.25 (1H, d, J 2.2 Hz), 7.74-7.72 (2H, m), 7.59-7.51 (5H, m), 7.27 (2H, t, J 8.6 Hz), 6.60 (1H, d, J 2.2 Hz), 6.39 (1H, s). m/z 305 (M+H, 100%), retention time 2.68 min (Method A).

### EXAMPLE 2

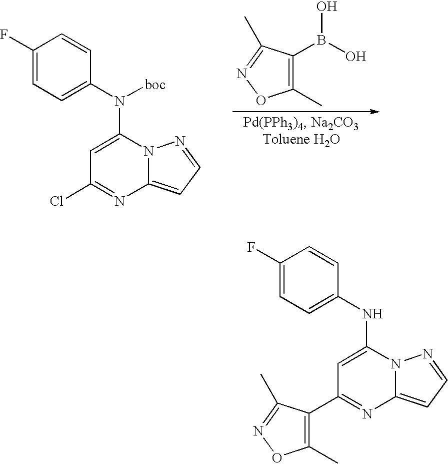

### 5-(3,5-Dimethylisoxazole)-7-(4-fluorophenylamino)pyrazolo[1,5-a]pyrimidine

To a solution of 5-chloro-7-(N-tert-butoxycarbonyl-4-fluorophenylamino)pyrazolo[1,5-a]pyrimidine (Example 1, Step 2) (0.05 g, 0.14 mmol) in 1,4-dioxane (3.5 cm³) and water (1 cm³) was added 3,5-dimethylisoxazole-4-boronic acid (0.023 g, 0.16 mmol) and sodium carbonate (0.031 g, 0.29 mmol). The solution was degassed by bubbling nitrogen through the mixture for 5 min. Tetrakis(triphenylphosphine)palladium(0) (0.015 g, 0.012 mmol) was added to the mixture and the reaction heated at 150° C. for 10 min in a microwave oven. The reaction mixture was concentrated in vacuo and purified on silica eluting with 2% methanol in dichloromethane to yield the title compound as a white solid (0.021 g, 47%).

δ_{H} (400 MHz; d-CHCl₃) 8.03 (1H, d, J 2.3 Hz), 7.97 (1H, s), 7.33-7.30 (2H, m), 7.13 (2H, t, J 8.5 Hz), 6.52 (1H, d, J 2.3 Hz), 6.13 (1H, s), 2.50 (3H, s), 2.34 (3H, s). m/z 324 (M+H, 100%), retention time 2.51 min (Method A).

### EXAMPLES 3-8

The compounds of Examples 3-8, listed in the following Table 1 were commercially available from BioFocus (BioFocus pic, Chesterford Park, Saffron Walden, Essex, CB10 1XL). The compounds of Examples 1 and 2 are also included in the Table. All compounds were tested for CDK2, CHK1 and PDK1 inhibitory activity in the assays described below in the Assay section. The result obtained in each case is given in the Table.

TABLE 1

| | | CDK2 | Chk1 | PDK1 | | RT |
|---|---|---|---|---|---|---|
| Structure | Example | IC₅₀(μM) | IC₅₀(μM) | IC₅₀(μM) | m/z | (min) |
| 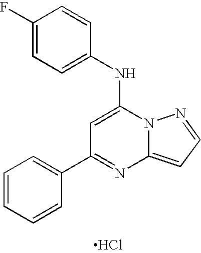 | 1 | 6.09 | >200 | >200 | 305 (M + H, 100%) | 2.68 |
| 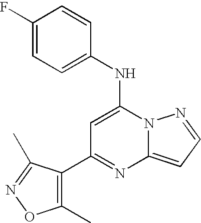 | 2 | 13.37 | >200 | >200 | 324 (M + H, 100%) | 2.51 |
| 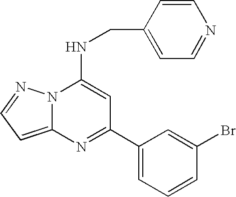 | 3 | 1.64 | 29.6 | 34.6 | 380 and 382 (each M + H, 100%) | 2.34 |
| 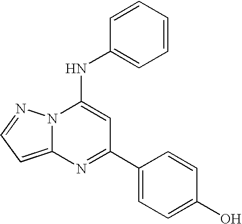 | 4 | 3.76 | >200 | >200 | 303 (M + H, 100%) | 2.20 |
| 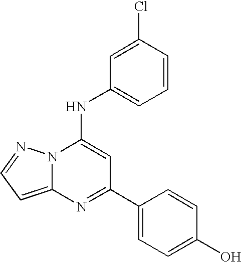 | 5 | 3.96 | >200 | >200 | 337 (M + H, 100%) | 2.42 |
| 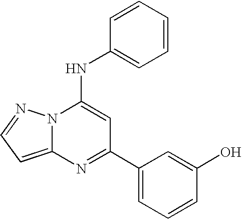 | 6 | 5.65 | >200 | >200 | 303 (M + H, 100%) | 2.32 |
| 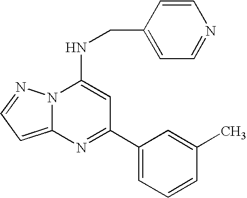 | 7 | 7.19 | >200 | >200 | 316 (M + H, 100%) | 2.07 |
| 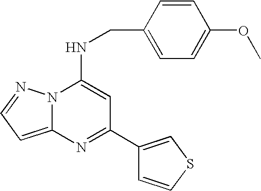 | 8 | 7.55 | >200 | >200 | 337 (M + H, 100%) | 2.48 |

The compounds of Examples 9-23, listed in the following Table 2 were prepared by methods analogous to those of Example 1. All compounds were tested for CDK2, CHK1 and PDK1 inhibitory activity in the assays described below in the Assay section. The result obtained in each case is given in the Table.

TABLE 2

| | | CDK2 | Chk1 | PDK1 | | RT |
|---|---|---|---|---|---|---|
| Structure | Example | IC₅₀(μM) | IC₅₀(μM) | IC₅₀(μM) | m/z | (min) |
| 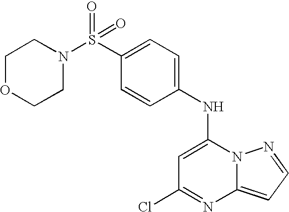 | 9 | 0.99 | >200 | >200 | 394 and 396 (each M + H, 100 and 35%) | 2.33 |
| 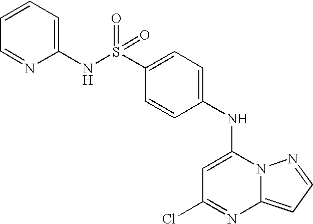 | 10 | 1.63 | >200 | >200 | 401 and 403 (each M + H, 100 and 35%) | 2.05 |
| 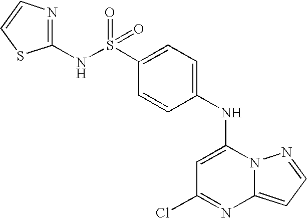 | 11 | 1.31 | >200 | >200 | 407 and 409 (each M + H, 100 and 35%) | 2.10 |
| 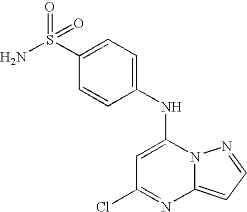 | 12 | 0.72 | >200 | >200 | 324 and 326 (each M + H, 100 and 35%) | 2.02 |
| 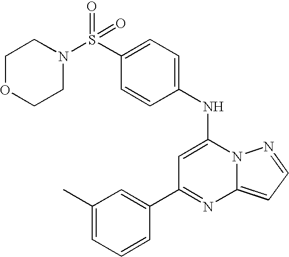 | 13 | 1.76 | >200 | >200 | 450 (M + H, 100%) | 2.66 |
| 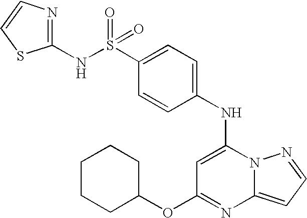 | 14 | 0.59 | >200 | 24.6 | 471 (M + H, 100%) | 2.63 |
| 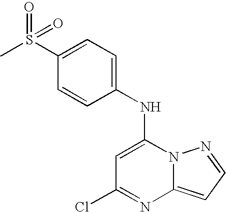 | 15 | 1.99 | >200 | >200 | 323 and 325 (each M + H, 100 and 35%) | 2.14 |
| 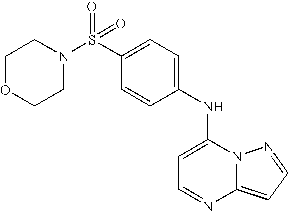 | 16 | 3.35 | >200 | >200 | 360 (M + H, 100%) | 1.98 |
| 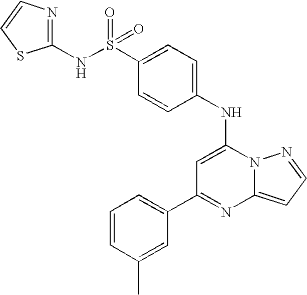 | 17 | 3.17 | >200 | 31.6 | 463 (M + H, 100%) | 2.36 |
| 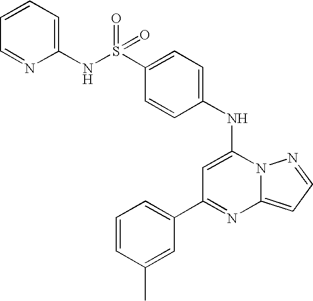 | 18 | 0.96 | 52.4 | >200 | 457 (M + H, 100%) | 2.36 |
| 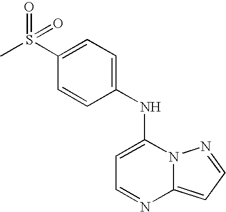 | 19 | 10.44 | ND | >200 | 289 (M + H, 100%) | 1.75 |
| 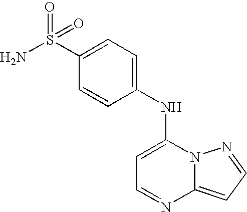 | 20 | 1.76 | >200 | >200 | 290 (M + H, 100%) | 1.59 |
| 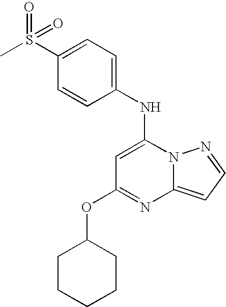 | 21 | 1.32 | >200 | >200 | 387 (M + H, 100%) | 273 |
| 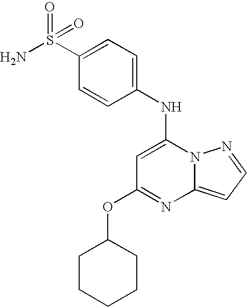 | 22 | 0.30 | >200 | 70.3 | 388 (M + H, 100%) | 2.61 |
| 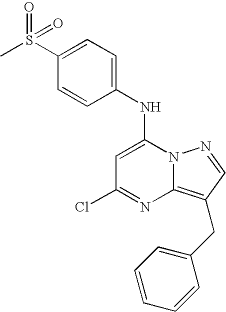 | 23 | >200 | >200 | >200 | 413 and 415 (each M + H, 100 and 35%) | 286 |

### EXAMPLES 24 AND 25

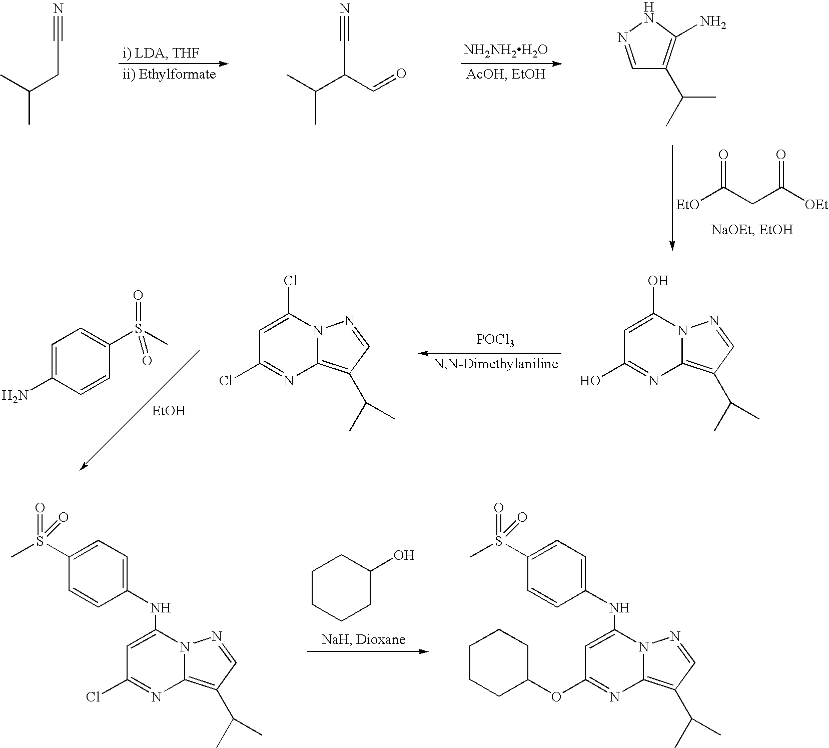

### Step 1

### 2-formyl-3-methylbutanenitrile

To a solution of diisopropyl amine (25.2 cm³, 0.180 mol) in tetrahydrofuan (100 cm³) at −78° C. was added dropwise n-butyllithium (1.6 M in hexanes, 112.8 cm³, 0.180 mol). The reaction was stirred at —78° C. for 30 min. Isovaleronitrile (18.9 cm³, 0.180 mol) was added and the reaction stirred for 10 min. The reaction mixture was added to a solution of ethyl formate (15.3 cm³, 0.190 mol) in tetrahydrofuran (50 cm³) at −78° C. The reaction was stirred at −78° C. for 30 min. then allowed to warm to room temperature and stirred for 16 h. The reaction was diluted with aqueous hydrochloric acid (300 cm³, 1M) until the pH was approximately pH=3. The product was extracted with ethyl acetate (3×100 cm³). The combined organic fractions were washed with brine then dried over magnesium sulphate and concentrated in vacuo. The product was purified on silica gel eluting with 50% diethyl ether in hexanes, to yield the title compound as a yellow oil (14.6 g, 73%).

δ_{H} (400 MHz; d-CHCl₃) 9.51 (1H, d, J 1.1 Hz), 3.35 (1H, dd, J 4.9, 1.0), 2.43-2.38 (1H, m), 1.12 (3H, d, J 6.6), 1.05 (3H, d, J 6.7).

### Step 2

### 3-amino-4-isopropylpyrazole

To a solution of 2-formyl-3-methyl-butanenitrile (9.47 g, 85.2 mmol) in ethanol (250 cm³) was added hydrazine hydrate (6.27 cm³, 110.8 mmol) and acetic acid (8.30 cm³, 144.8 mmol). The reaction was heated under reflux for 16 h. The reaction was concentrated in vacuo to approximately one third the original volume. The residue was diluted with aqueous sodium bicarbonate (100 cm³, saturated solution) and the product extracted with dichloromethane (3×100 cm³). The combined organic fractions were washed with brine then dried over magnesium sulphate and concentrated in vacuo to yield the crude product as a brown solid (9.35 g, 88%).

δ_{H} (400 MHz; d-CHCl₃) 6.99 (1H, s), 2.55 (1H, sept, J 6.8), 1.06 (6H, d, J 6.8). m/z 126 (M+H, 100%), retention time 1.21 min (Method A).

### Step 3

### 3-isopropyl-5,7-dihydroxypyrazolo[1,5-a]pyrimidine

Sodium (0.98 g, 42.8 mmol) was dissolved in ethanol (200 cm³) and to the solution was added 3-amino-4-isopropyl-pyrazole (4.46 g, 35.6 mmol) and diethyl malonate (5.95 cm³, 39.2 mmol). The reaction was heated under reflux for 16 h. The reaction was concentrated in vacuo and the residue dissolved in water (50 cm³). The reaction was acidified to approx pH=3 with hydrochloric acid (2N) and the precipitate formed was collected by filtration. The solid was washed with water (3×50 cm³) and dried in vacuo to yield the product as an off-white solid (3.95 g, 57%).

δ_{H} (400 MHz; d₆-DMSO) 7.94 (1H, s), 7.84 (1H, s), 5.06 (1H, s), 3.91 (2H, s), 3.23-3.08 (2H, m), 1.32 (6H, d, J 6.8), 1.30 (6H, d, J 6.8). m/z 194 (M+H, 100%), retention time 1.38 min (Method A).

### Step 4

### 3-isopropyl-5,7-dichloropyrazolo[1,5-a]pyrimidine

3-isopropyl-5,7-dihydroxypyrazolo[1,5-a]pyrimidine (3.95 g, 20.4 mmol) and N,N-dimethylaniline (1.73 cm³, 13.6 mmol) were suspended in phosphorous oxychloride (38.1 cm³, 0.41 mol). The reaction was heated under reflux for 16 h, over which time the 3-isopropyl-5,7-dihydroxypyrazolo[1,5-a]pyrimidine dissolved. The reaction was concentrated in vacuo and the residue poured onto ice (approx 50 g). The product was extracted with dichloromethane (3×50 cm³). The combined organic fractions were washed with brine then dried over magnesium sulphate and concentrated in vacuo. The product was purified on silica eluting with 5% ethyl acetate in hexanes, to yield the title compound as a yellow solid (3.90 g, 83%).

δ_{H} (400 MHz; d-CHCl₃) 7.92 (1H, s), 6.74 (1H, s), 3.14 (1H, sept, J 6.9), 1.19 (6H, d, J 6.9). m/z 230 and 232 each (M+H, 100% and 65%), retention time 2.65 min (Method A).

### Step 5

### 3-isopropyl-5-chloro-7-(4-methylsulphonylaminophenyl)pyrazolo[1,5-a]pyrimidine (Example 24)

To a solution of 3-isopropyl-5,7-dichloropyrazolo[1,5-a]pyrimidine (0.50 g, 2.17 mmol) in ethanol (20 cm³) was added 4-methylsulphonylaniline (0.50 g, 2.39 mmol). The reaction was heated under reflux for 16 h. The reaction was concentrated in vacuo and the residue triturated with hot methanol (2×10 cm³) to yield the product as a white solid (0.56 g, 70%).

δ_{H} (400 MHz; d₆-DMSO) 10.53 (1H, s), 8.05 (1H, s), 7.85 (2H, d, J 6.8), 7.60 (2H, d, J 6.8), 6.28 (1H, s), 3.11 (3H, s), 3.02 (1H, sept, J 6.9), 1.18 (6H, d, J 6.9). m/z 365 and 367 each (M+H, 100% and 35%), retention time 2.57 min (Method A).

### Step 6

### 3-isopropyl-5-cyclohexanyloxy-7-(4-methylsulphonylaminophenyl)pyrazolo[1,5-a]pyrimidine (Example 25)

To a solution of cyclohexanol (0.14 cm³, 1.37 mmol) in dioxane (5 cm³) was added sodium hydride (0.11 g, 60% by wt in oil, 2.74 mmol). Once effervescence had ceased 3-isopropyl-5-chloro-7-(4-methylsulphonylaminophenyl)pyrazolo[1,5-a]pyrimidine (0.10 g, 0.27 mmol) was added. The reaction was heated via a microwave reactor, in a sealed tube, at 120° C. for 20 min. The reaction was poured into water (20 cm³) and the product extracted with ethyl acetate (3×20 cm³). The combined organic fractions were dried with brine then magnesium sulphate and concentrated in vacuo. The product was purified on silica eluting with 25-50% ethyl acetate in hexanes, to yield the title compound as a white solid (0.008 g, 7%).

δ_{H} (400 MHz; d-CDCl₃) 8.18 (1H, s), 7.98 (2H, d, J 6.8), 7.78 (1H, s), 7.48 (2H, d, J 6.8), 5.17-5.13 (1H, m), 3.15 (1H, sept, J 6.8), 3.07 (3H, s), 2.04-2.02 (2H, m), 1.80-1.77 (2H, m),1.60-1.43 (6H, m), 1.35 (6H, d, J 6.9). m/z 429 (M+H, 100%), retention time 3.05 min (Method A).

The compounds of Examples 26-28, listed in the following Table 3 were prepared by methods analogous to those of Examples 24 and 25. The compounds of Examples 24 and 25 are also included in the Table. All compounds were tested for CDK2 inhibitory activity in the assay described below in the Assay section. The result obtained in each case is given in the Table 3.

TABLE 3

| | | CDK2 IC₅₀ | | |
|---|---|---|---|---|
| Structure | Example | (μM) | m/z | RT (min) |
| 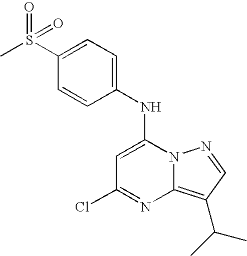 | 24 | 0.22 | 365 and 367 (each M + H, 100 and 35%) | 2.58 |
| 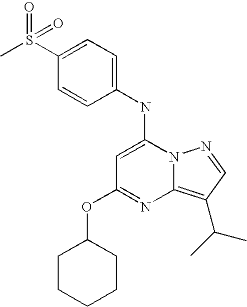 | 25 | 0.95 | 429 (M + H, 100%) | 305 |
| 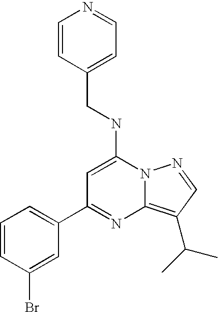 | 26 | 0.53 | 424 and 426 (each M + H, 100%) | 2.67 |
| 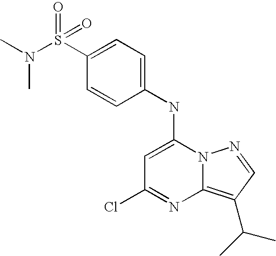 | 27 | 0.24 | 394 and 396 (each M + H, 100 and 35%) | 2.81 |
| 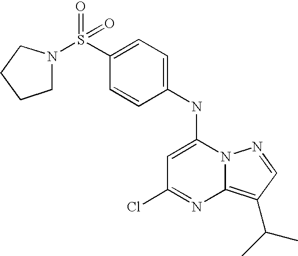 | 28 | 0.27 | 401 and 403 (each M + H, 100 and 35%) | 2.05 |

### EXAMPLE 29

### 3-isopropyl-5-chloro-7-(4-(N,N-dimethylsulphonamido)phenylamino)pyrazolo[1,5-a]pyrimidine

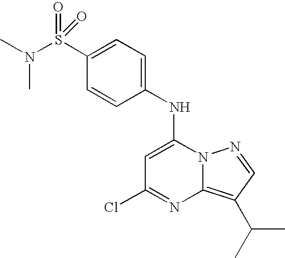

To a solution of 3-isopropyl-5,7-dichloropyrazolo[1,5-a]pyrimidine (0.15 g, 0.66 mmol) in ethanol (20 cm³) was added 4-amino-N,N-dimethylbenzenesulphonamide (0.146 g, 0.73 mmol). The reaction was heated at reflux for 16 h. The reaction was concentrated in vacuo and the residue triturated with hot ethanol (2×10 cm³) to yield the product as a white solid (0.23 g, 92%).

δ_{H} (400 MHz; d₆-DMSO) 10.41 (1H, s), 7.97 (1H, s), 7.59 (2H, d, J 6.7), 7.52 (2H, d, J 6.7), 6.26 (1H, s), 2.94 (1H, sept, J 6.9), 2.43 (6H, s), 1.10 (6H, d, J 6.9). m/z 394 and 396 each (M+H, 100% and 35%), retention time 2.78 min (Method A).

### EXAMPLE 30

### 3-isopropyl-5-(trans-4-aminocyclohexylamino)-7-(4-(N,N-dimethylsulphonamido)phenylamino)pyrazolo[1,5-a]pyrimidine

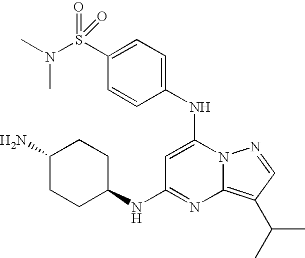

To a solution of 3-isopropyl-5-chloro-7-(4-(N,N-dimethylsulphonamido)phenylamino)pyrazolo[1,5-a]pyrimidine (0.40 g, 1.02 mmol) in dioxane (3 cm³) was added acetonitrile (1 cm³), 1,4-trans-diaminocyclohexane (1.17 g, 10.24 mmol) and triethylamine (0.71 cm³, 5.12 mmol). The reaction was heated via a microwave, in a sealed tube, at 180° C. for 2 hours. The reaction mixture was loaded onto a silica flash column and the product eluted with with 15% methanol in dichloromethane, to yield the title compound as a white solid (0.088 g, 18%).

δ_{H} (400 MHz; d₄-CDCl₃) 8.00 (1H, s), 7.74 (2H, d, J 6.7), 7.64 (1H, s), 7.36 (2H, d, J 6.7), 5.67 (1H, s), 4.41 (1H, d, J 7.8), 3.42-3.40 (1H, m), 3.05 (1H, sept, J 6.9), 2.89-2.81 (1H, m), 2.15 (2H, d, J 10.9), 1.93 (2H, d, J 9.2), 2.11-1.62 (2H, br s), 1.47-1.39 (2H, m), 1.27 (6H, d, J 6.9), 1.25-1.15 (2H, m). m/z 472 (M+H, 100%), retention time 1.93 min (Method A).

### EXAMPLE 31

### Step 1

### 3-bromo-5,7-chloropyrazolo[1,5-a]pyrimidine

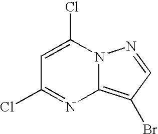

To a solution of 5,7-chloropyrazolo[1,5-a]pyrimidine (1 g, 5.32 mmol) in acetonitrile (20 cm³) was added N-bromosuccinimide (1.04 g, 5.85 mmol) and ceric ammoinum nitrate (0.029 g, 0.053 mmol). The reaction was heated at reflux for 1 hour. The reaction was washed with aqueous sodium metabisulfite (30 cm³, 10% solution) and then brine (20 cm³). The organic fraction was dried with magnesium sulphate and concentrated in vacuo. The product was purified on silica eluting with 20% ethylacetate in hexane, to yield the title compound as a yellow solid (1.33 g, 92%).

δ_{H} (400 MHz; d₄-CDCl₃) 8.22 (1H, s), 7.04 (1H, s).

### Step 2

### 3-bromo-5-chloro-7-(4-(N,N-dimethylsulphonamido)phenylamino)pyrazolo[1,5-a]pyrimidine

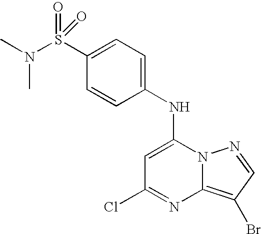

To a solution of 3-bromo-5,7-dichloropyrazolo[1,5-a]pyrimidine (0.14 g, 0.53 mmol) in ethanol (20 cm³) was added 4-amino-N,N-dimethbenzenesulphonamide (0.107 g, 0.53 mmol). The reaction was heated at reflux for 16 h. The reaction was concentrated in vacuo and the residue triturated with hot ethanol (2×10 cm³) to yield the product as a white solid (0.10 g, 43%).

δ_{H} (400 MHz; d₄-CDCl₃) 8.10 (1H, s), 7.89 (2H, d, J 6.7), 7.66 (2H, d, J 6.7), 6.51 (1H, s), 2.74 (6H, s). m/z 430, 432 and 434 each (M+H, 75%, 100% and 25%), retention time 2.58 min (Method A).

### Step 3

### 3-bromo-5-(trans-4-aminocyclohexylamino)-7-(4-(N,N-dimethylsulphonamido)phenylamino)pyrazolo[1,5-a]pyrimidine

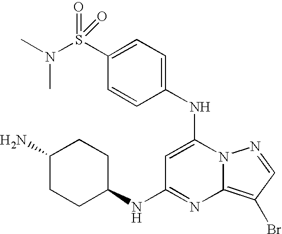

To a solution of 3-bromo-5-chloro-7-(4-(N,N-dimethylsulphonamido)phenylamino)pyrazolo[1,5-a]pyrimidine (0.05 g, 0.12 mmol) in dioxane (3 cm³) was added acetonitrile (1 cm³), 1,4-trans-diaminocyclohexane (0.13 g, 1.16 mmol) and triethylamine (0.08 cm³, 0.58 mmol). The reaction was heated via a microwave, in a sealed tube, at 180° C. for 2 hours. The reaction mixture was loaded onto a silica flash column and the product eluted with with 20% methanol in dichloromethane, to yield the title compound as a white solid (0.05 g, 82%).

δ_{H} (400 MHz; d-MeOH) 7.74 (2H, d, J 6.7), 7.71 (1H, s), 7.52 (2H, d, J 6.7), 5.89 (1H, s), 3.97-3.83 (1H, m), 2.92-2.83 (1H, m), 2.12 (2H, d, J 10.92), 1.96 (2H, d, J 12.6),1.44-1.38 (2H, m), 1.29-1.20 (2H, m). m/z 510 and 512 (M+H, 100% and 100%), retention time 1.90 min (Method A).

The compounds of Examples 29-31 were tested, together with additional compounds synthesised by methods analogous to those of Examples 29-31, in the assays described below in the Assay section. The result obtained in each case is given in the following Table 4.

TABLE 4

| | | CDK2 | Chk1 | PDK1 | | RT |
|---|---|---|---|---|---|---|
| Structure | Example | IC₅₀(μM) | IC₅₀(μM) | IC₅₀(μM) | m/z | (min) |
| 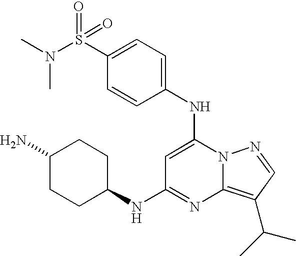 | 30 | 0.059 | 5.513 | 19.938 | 472 M + H, 100%) | 1.89 |
| 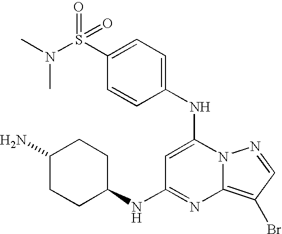 | 31 | 0.008 | 1.269 | 0.325 | 508 and 510 (each M + H, 100%) | 1.90 |
| 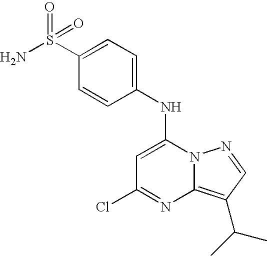 | 32 | 0.062 | >200 | >200 | 366 and 368 (each M + H, 100 and 35%) | 2.69 |
| 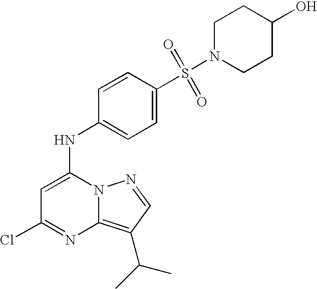 | 33 | 0.271 | >200 | >200 | 450 and 452 (each M + H, 100 and 35%) | 2.44 |
| 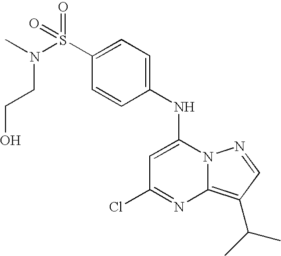 | 34 | 0.127 | >200 | >200 | 424 and 426 (each M + H, 100 and 35%) | 2.72 |
| 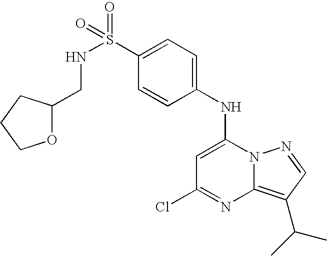 | 35 | 0.191 | >200 | >200 | 450 and 452 (each M + H, 100 and 35%) | 2.87 |
| 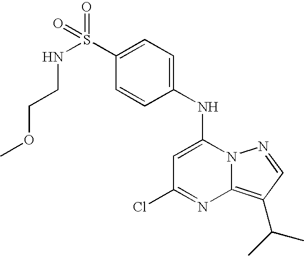 | 36 | 0.715 | >200 | >200 | 424 and 426 (each M + H, 100 and 35%) | 2.81 |
| 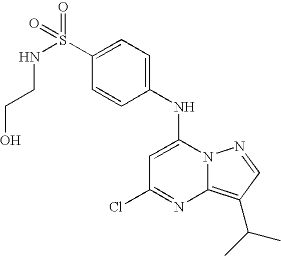 | 37 | 1.513 | >200 | >200 | 410 and 412 (each M + H, 100 and 35%) | 2.61 |
| 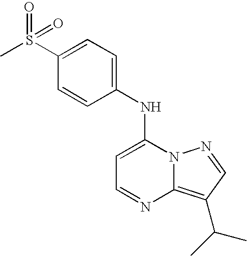 | 38 | 0.313 | >200 | >200 | 331 (M + H, 100%) | 2.25 |
| 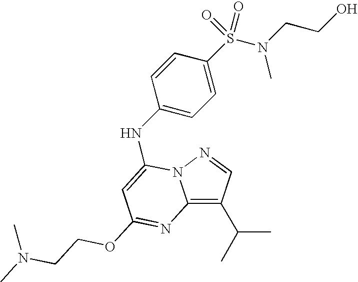 | 39 | 0.403 | 80.776 | >200 | 477 (M + H, 100%) | 1.88 |
| 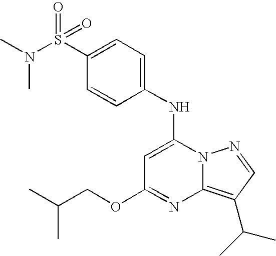 | 40 | 1.164 | >200 | >200 | 432 (M + H, 100%) | 3.08 |
| 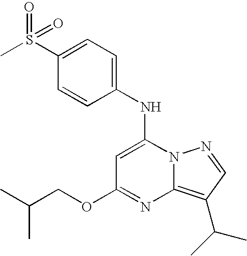 | 41 | 1.196 | >200 | >200 | 403 (M + H, 100%) | 2.97 |
| 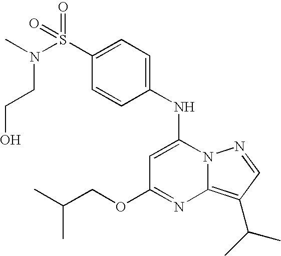 | 42 | 0.170 | >200 | >200 | 462 (M + H, 100%) | 2.92 |
| 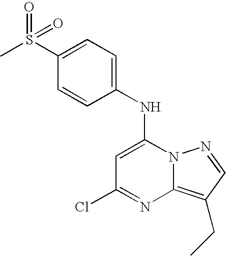 | 43 | 0.107 | >200 | >200 | 351 and 353 (each M + H, 100 and 35%) | 2.48 |
| 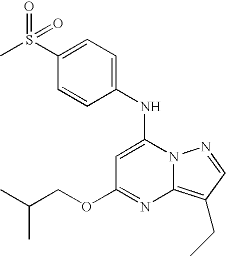 | 44 | 0.179 | >200 | 79.095 | 389 (M + H, 100%) | 2.88 |
| 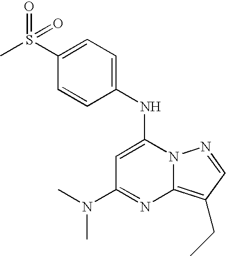 | 45 | 1.134 | >200 | >200 | 360 (M + H, 100%) | 2.43 |
| 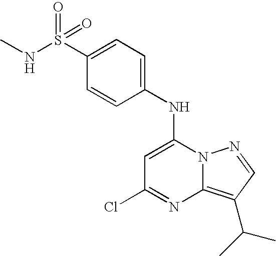 | 46 | 0.076 | >200 | 108.96 | 380 and 382 (each M + H, 100 and 35%) | 2.64 |
| 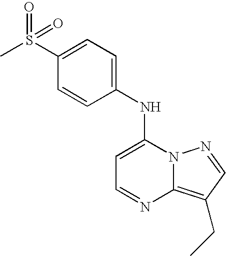 | 47 | 0.727 | >200 | >200 | 317 (M + H, 100%) | 2.07 |
| 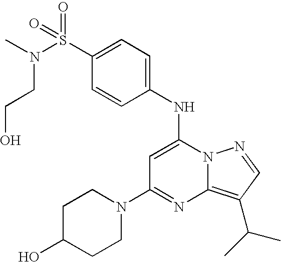 | 48 | 1.455 | >200 | >200 | 489 (M + H, 100%) | 2.31 |
| 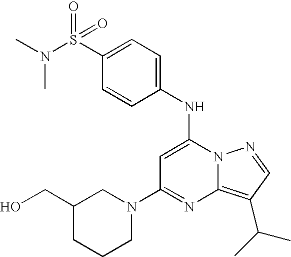 | 49 | 0.387 | >200 | >200 | 473 (M + H, 100%) | 2.68 |
| 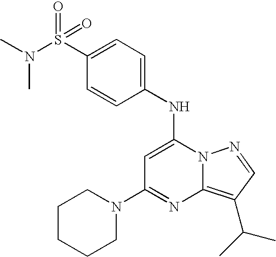 | 50 | 1.009 | >200 | >200 | 443 (M + H, 100%) | 2.98 |
| 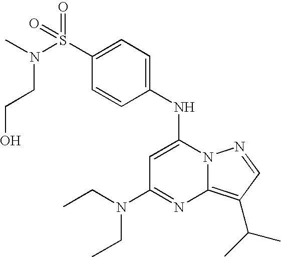 | 51 | 0.259 | >200 | >200 | 461 (M + H, 100%) | 2.82 |
| 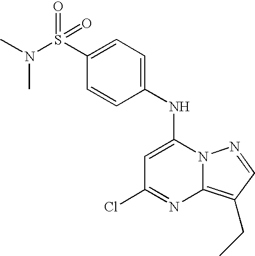 | 52 | 0.239 | >200 | >200 | 380 and 382 (each M + H, 100 and 35%) | 2.65 |
| 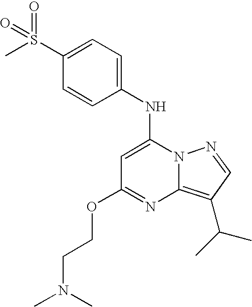 | 53 | 8.415 | >200 | >200 | 418 (M + H, 100%) | 1.87 |
| 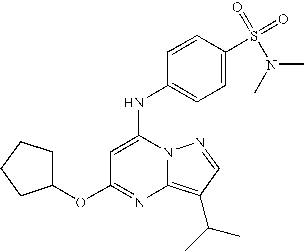 | 54 | 1.308 | >200 | >200 | 444 (M + H, 100%) | 2.80 |
| 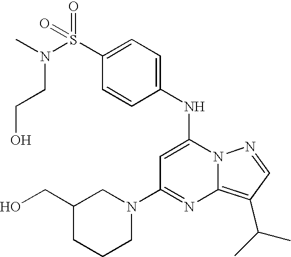 | 55 | 0.270 | >200 | >200 | 503 (M + H, 100%) | 2.47 |
| 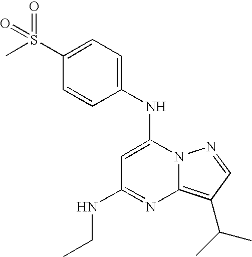 | 56 | 0.850 | >200 | 45.334 | 374 (M + H, 100%) | 2.41 |
| 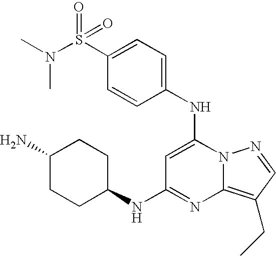 | 57 | 0.069 | 2.611 | 2.436 | 458 (M + H, 100%) | 1.79 |
| 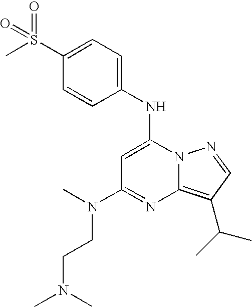 | 58 | 1.945 | 51.735 | >200 | 431 (M + H, 100%) | 1.87 |
| 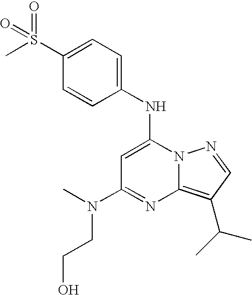 | 59 | 0.768 | >200 | >200 | 404 (M + H, 100%) | 2.27 |
| 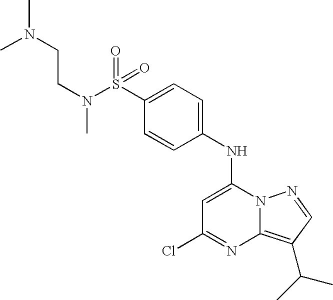 | 60 | 0.040 | >200 | >200 | 451 and 453 (each M + H, 100 and 35%) | 2.10 |
| 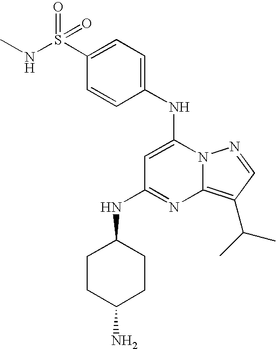 | 61 | 0.032 | 6.634 | 11.015 | 458 (M + H, 100%) | 1.83 |
| 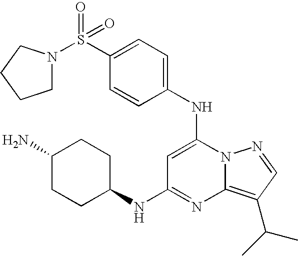 | 62 | 0.024 | 3.240 | 1.196 | 498 (M + H, 100%) | 2.00 |
| 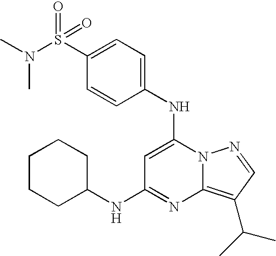 | 63 | 0.355 | >200 | >200 | 457 (M + H, 100%) | 2.97 |
| 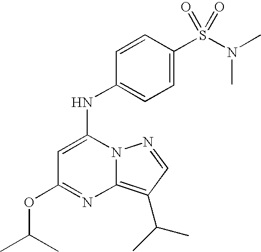 | 64 | 0.395 | >200 | >200 | 418 (M + H, 100%) | 3.00 |
| 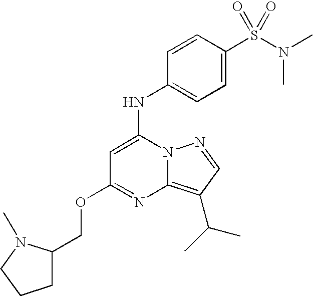 | 65 | 1.091 | >200 | >200 | 473 (M + H, 100%) | 2.06 |
| 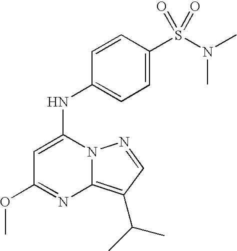 | 66 | 0.876 | >200 | >200 | 390 (M + H, 100%) | 2.82 |
| 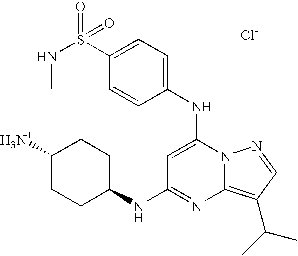 | 67 | 0.030 | >200 | 4.636 | 458 (M + H, 100%) | 1.81 |
| 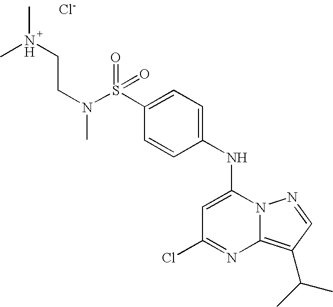 | 68 | 0.052 | >200 | >200 | 451 and 453 (each M + H, 100 and 35%) | 2.10 |
| 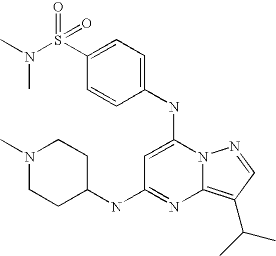 | 69 | 0.181 | 19.25 | 31.630 | 472 (M + H, 100%) | 2.00 |
| 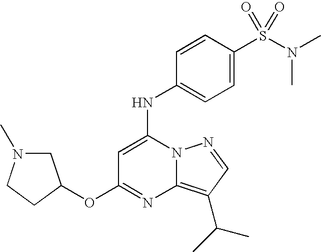 | 70 | 0.320 | 79.860 | >200 | 459 (M + H, 100%) | 2.03 |
| 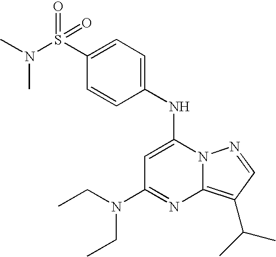 | 71 | 0.655 | >200 | >200 | 431 (M + H, 100%) | 2.96 |
| 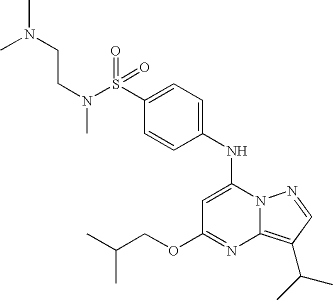 | 72 | 1.279 | 8.401 | >200 | 489 (M + H, 100%) | 2.48 |
| 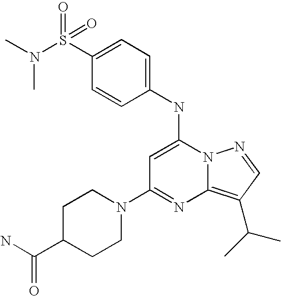 | 73 | 1.482 | >200 | >200 | 486 (M + H, 100%) | 2.41 |
| 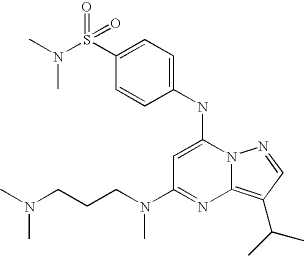 | 74 | 7.275 | 18.550 | >200 | 474 (M + H, 100%) | 2.03 |
| 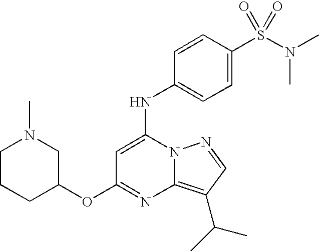 | 75 | 0.266 | 6.281 | >200 | 473 (M + H, 100%) | 2.07 |
| 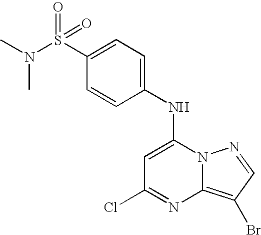 | 76 | 0.128 | >200 | >200 | 430, 432 and 434 (each M + H, 75, 100 and 25%) | 2.58 |
| 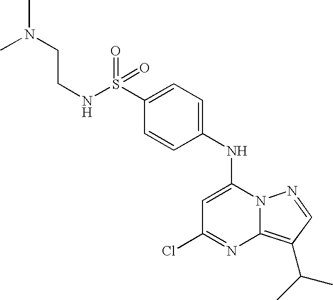 | 77 | 0.039 | >200 | 9.491 | 437 and 439 (each M + H, 100 and 35%) | 2.04 |
| 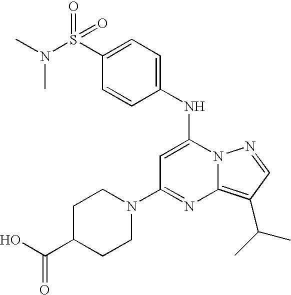 | 78 | 4.786 | >200 | >200 | 487 (M + H, 100%) | 2.59 |
| 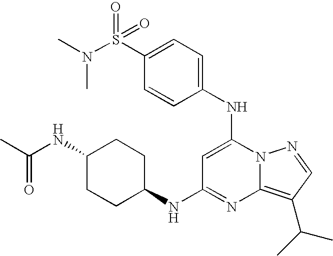 | 79 | 0.066 | >200 | >200 | 514 (M + H, 100%) | 2.32 |
| 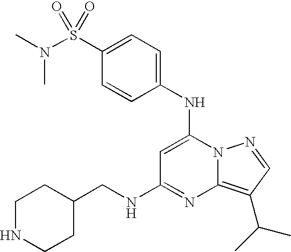 | 80 | 0.682 | >200 | 2.557 | 472 (M + H, 100%) | 1.95 |
| 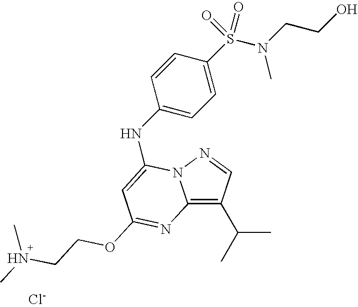 | 81 | 0.240 | 46.753 | >200 | 478 (M + H, 100%) | 1.85 |
| 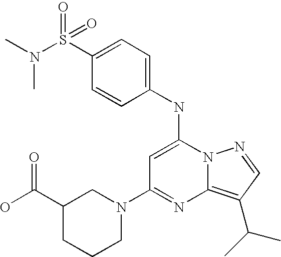 | 82 | 4.230 | >200 | >200 | 487 (M + H, 100%) | 2.58 |
| 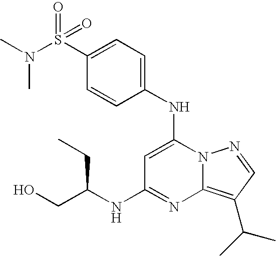 | 83 | 0.140 | >200 | >200 | 447 (M + H, 100%) | 2.52 |
| 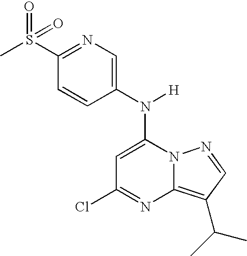 | 84 | 0.298 | >200 | >200 | 366 and 368 (each M + H, 100 and 35%) | 2.45 |
| 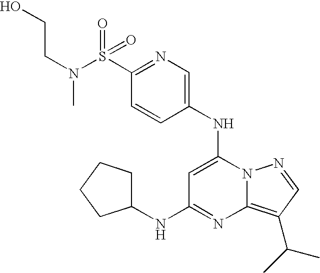 | 85 | 0.302 | >200 | >200 | 473 (M + H, 100%) | 2.73 |
| 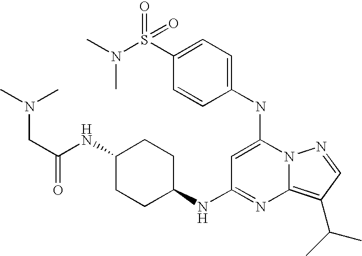 | 86 | 0.030 | >200 | >200 | 557 (M + H, 100%) | 1.98 |
| 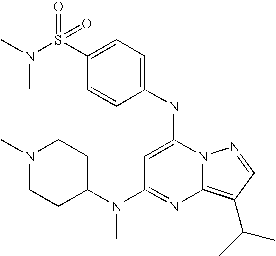 | 87 | 0.840 | 16.328 | >200 | 486 (M + H, 100%) | 2.06 |
| 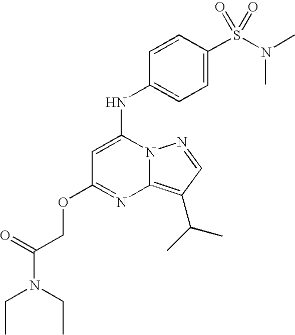 | 88 | 0.419 | >200 | >200 | 489 (M + H, 100%) | 2.68 |
| 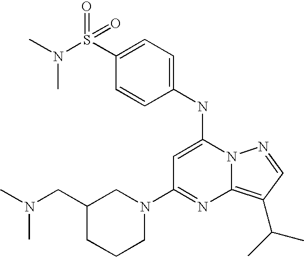 | 89 | 8.269 | >200 | >200 | 500 (M + H, 100%) | 2.09 |
| 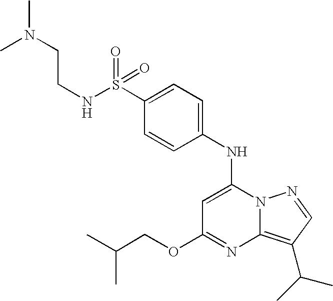 | 90 | 0.373 | 5.422 | >200 | 475 (M + H, 100%) | 2.38 |
| 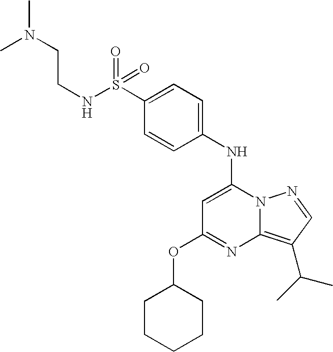 | 91 | 0.881 | 5.358 | >200 | 501 (M + H, 100%) | 2.56 |
| 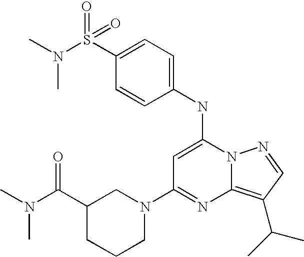 | 92 | 10.513 | >200 | >200 | 514 (M + H, 100%) | 2.90 |
| 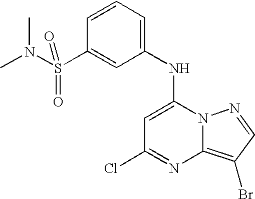 | 93 | 0.572 | >200 | >200 | 430, 432 and 434 (each M + H, 75, 100 and 25%) | 2.60 |
| 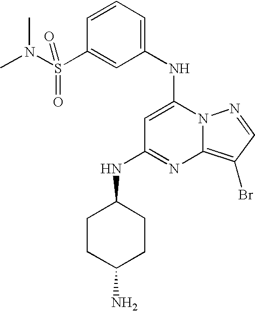 | 94 | 0.070 | 27.010 | >200 | 508 and 510 (each M + H, 100%) | 1.81 |
| 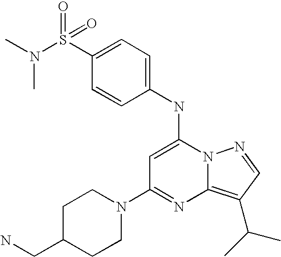 | 95 | 1.863 | 24.872 | 64.171 | 472 (M + H, 100%) | 2.05 |
| 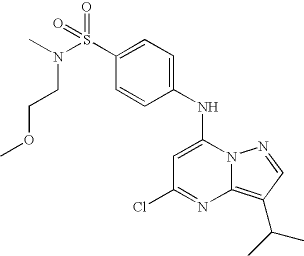 | 96 | 0.124 | >200 | >200 | 438 and 440 (each M + H, 100 and 35%) | 2.78 |
| 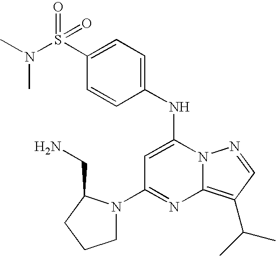 | 97 | 7.815 | 4.751 | >200 | 458 (M + H, 100%) | 2.00 |
| 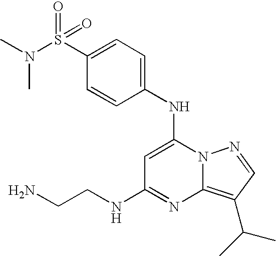 | 98 | 7.505 | 18.67 | >200 | 418 (M + H, 100%) | 1.91 |
| 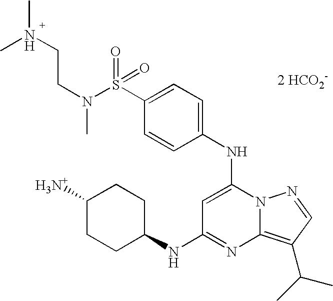 | 99 | 0.128 | 0.953 | 6.909 | 529 (M + H, 100%) | 1.66 |
| 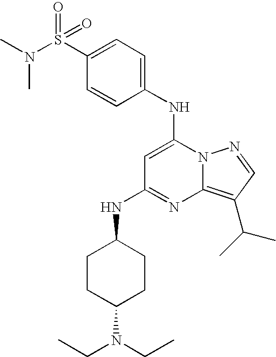 | 100 | 0.092 | 29.345 | 30.27 | 528 (M + H, 100%) | 2.09 |
| 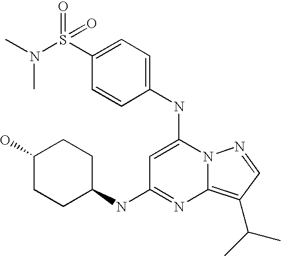 | 101 | 0.024 | >200 | 3.812 | 473 (M + H, 100%) | 2.35 |
| 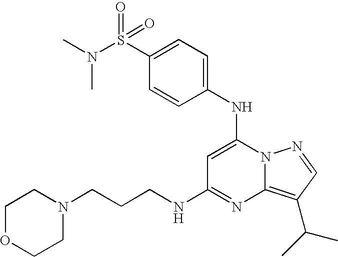 | 102 | 0.965 | >200 | >200 | 502 (M + H, 100%) | 2.00 |
| 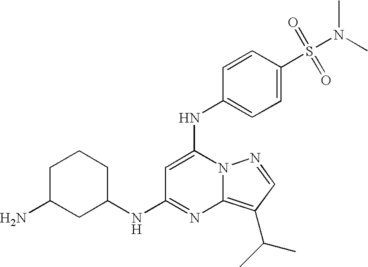 | 103 | 0.033 | 0.412 | 27.549 | 508 and 510 (each M + H, 100%) | 1.96 |
| 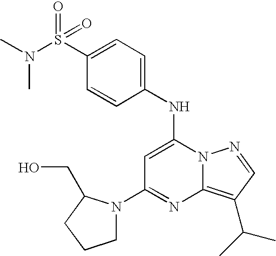 | 104 | 0.177 | >200 | >200 | 459 (M + H, 100%) | 2.49 |
| 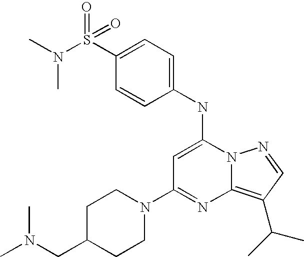 | 105 | 5.98 | >200 | >200 | 500 (M + H, 100%) | 2.07 |
| 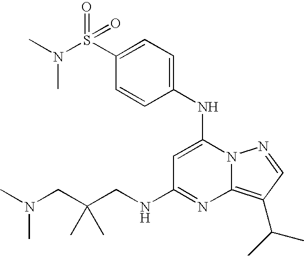 | 106 | 3.335 | 18.246 | 14.408 | 488 (M + H, 100%) | 2.11 |
| | | CDK2 | CHK1 | PDK1 | | RT |
| Structure | Example | IC₅₀(μM) | IC₅₀(μM) | IC₅₀(μM) | m/z | (min) |
| 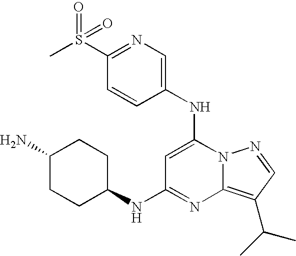 | 107 | 0.101 | 19.586 | 96.508 | 444 (M + H, 100%) | 1.74 |
| 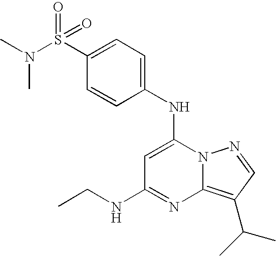 | 108 | 3.143 | >200 | >200 | 403 (M + H, 100%) | 2.61 |
| 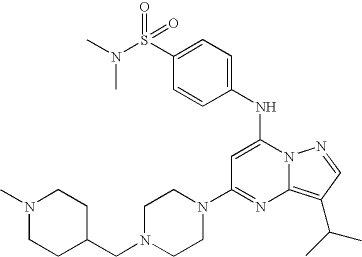 | 109 | 10.310 | 15.221 | >200 | 555 (M + H, 100%) | 1.82 |
| 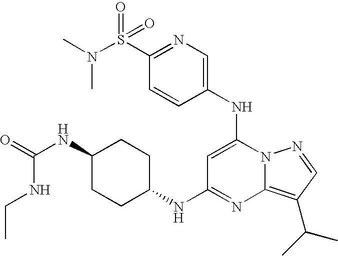 | 110 | 0.305 | >200 | >200 | 543 (M + H, 100%) | 2.36 |
| 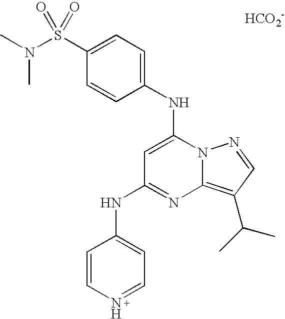 | 111 | 3.088 | >200 | >200 | 462 (M + H, 100%) | 2.08 |
| 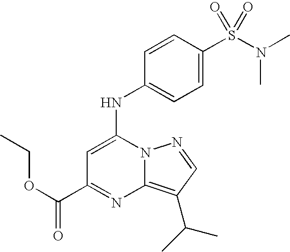 | 112 | 0.274 | >200 | >200 | 432 (M + H, 100%) | 2.69 |
| 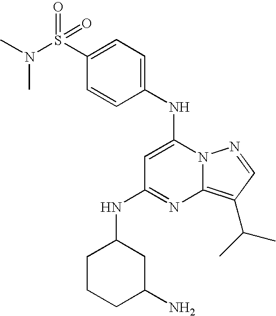 | 113 | 2.054 | 2.200 | 3.118 | 472 (M + H, 100%) | 2.00 |
| 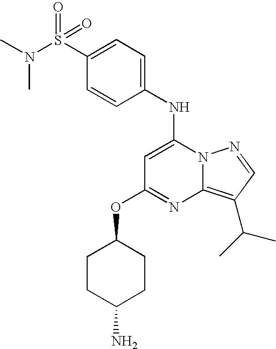 | 114 | 0.017 | 3.450 | 125.194 | 473 (M + H, 100%) | 2.06 |
| 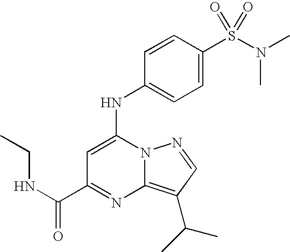 | 115 | 7.889 | >200 | >200 | 431 (M + H, 100%) | 2.67 |
| 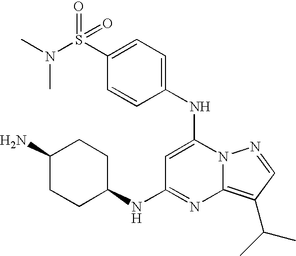 | 116 | 0.975 | 2.190 | 0.536 | 472 (M + H, 100%) | 2.01 |
| 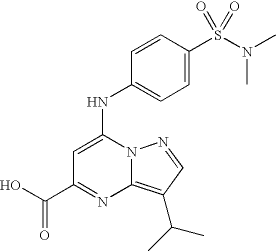 | 117 | 23.268 | >200 | >200 | 404 (M + H, 100%) | 2.27 |
| 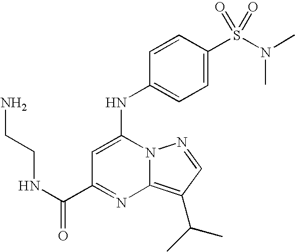 | 118 | 6.760 | 36.841 | >200 | 446 (M + H, 100%) | 1.91 |
| 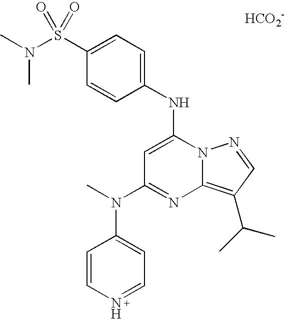 | 119 | 22.674 | >200 | >200 | 466 (M + H, 100%) | 2.11 |
| 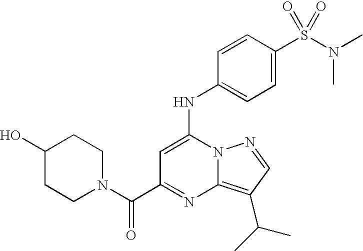 | 120 | 0.594 | >200 | >200 | 487 (M + H, 100%) | 2.19 |
| 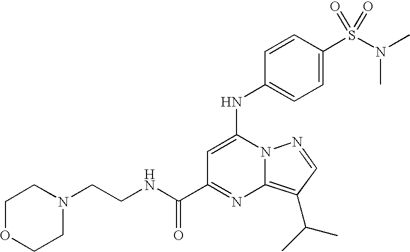 | 121 | 9.302 | >200 | >200 | 516 (M + H, 100%) | 1.96 |
| 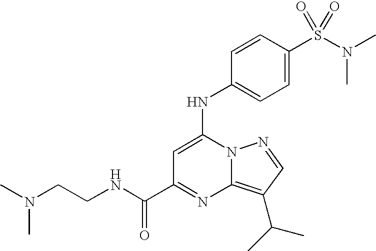 | 122 | 3.172 | >200 | >200 | 474 (M + H, 100%) | 1.93 |
| 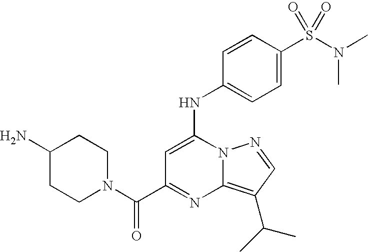 | 123 | 1.341 | >200 | 767.778 | 486 (M + H, 100%) | 1.82 |
| 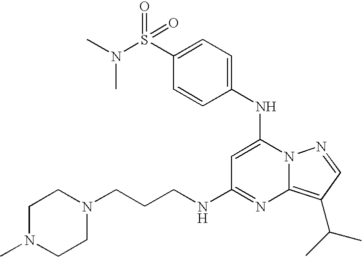 | 124 | 22.076 | >200 | >200 | 515 (M + H, 100%) | 1.97 |
| 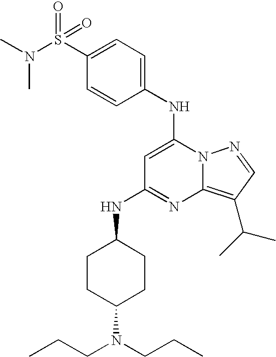 | 125 | 0.155 | 20.525 | 19.869 | 556 (M + H, 100%) | 2.14 |
| 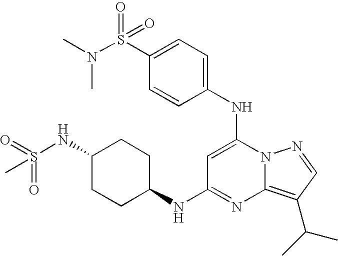 | 126 | 0.073 | >200 | 34.430 | 550 (M + H, 100%) | 2.52 |
| 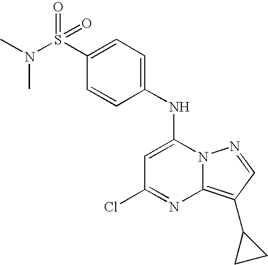 | 127 | 0.005 | >200 | 29.345 | 392 and 394 (each M + H, 100 and 35%) | 2.67 |
| 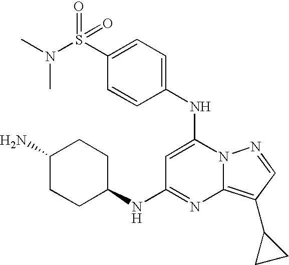 | 128 | 0.022 | 1.026 | 0.178 | 470 (M + H, 100%) | 1.89 |
| 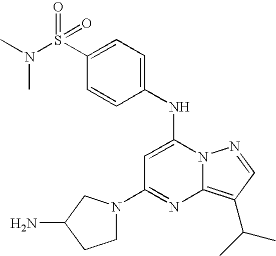 | 129 | 1.973 | ND | ND | 444 (M + H, 100%) | 1.95 |
| 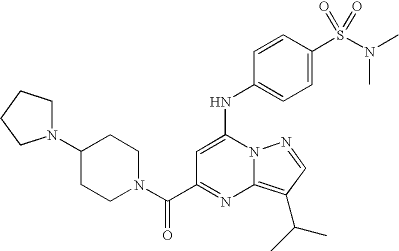 | 130 | 12.152 | ND | ND | 540 (M + H, 100%) | 1.86 |
| 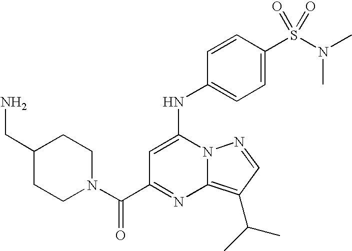 | 131 | 9.368 | ND | ND | 500 (M + H, 100%) | 1.84 |
| 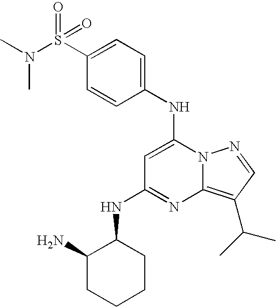 | 132 | 3.628 | ND | 10.371 | 472 (M + H, 100%) | 2.10 |
| 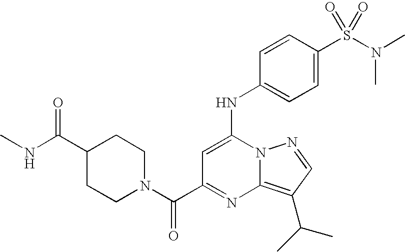 | 133 | 5.169 | ND | ND | 528 (M + H, 100%) | 2.16 |
| 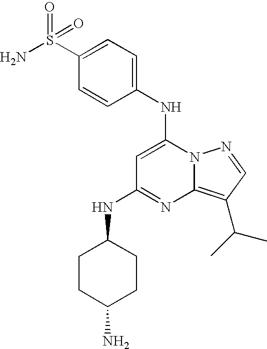 | 134 | 0.026 | ND | 4.429 | 444 (M + H, 100%) | 1.73 |
| 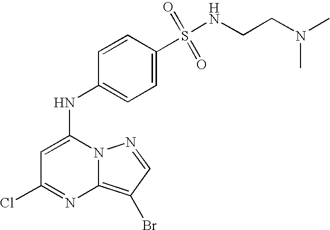 | 135 | 0.081 | 43 | >50 | 473, 475 and 477 (each M + H, 75, 100 and 25%) | 1.86 |
| 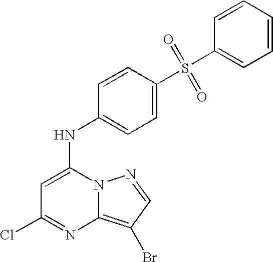 | 136 | 0.204 | ND | 16 | 463, 465 and 467 (each M + H, 75, 100 and 25%) | 2.71 |
| 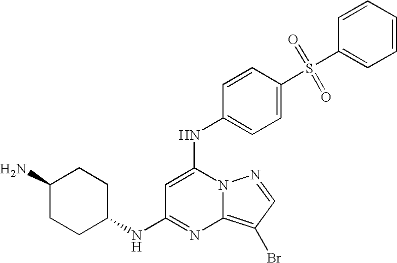 | 137 | 0.016 | 0.717 | 1.057 | 541 and 543 (each M + H, 100%) | 2.00 |
| 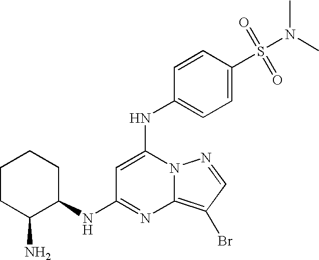 | 138 | 0.565 | 0.97 | >50 | 508 and 510 (each M + H, 100%) | 1.99 |
| 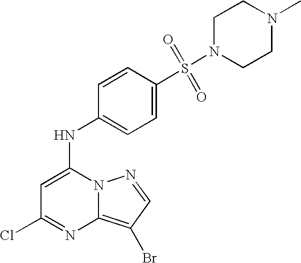 | 139 | 0.176 | ND | >50 | 485, 487 and 489 (each M + H, 75, 100 and 25%) | 1.96 |
| 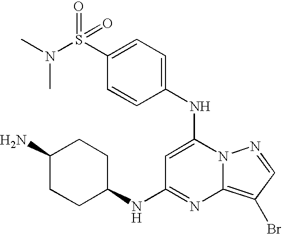 | 140 | 3.657 | 0.500 | 5.287 | 508 and 510 (each M + H, 100%) | 1.94 |
| 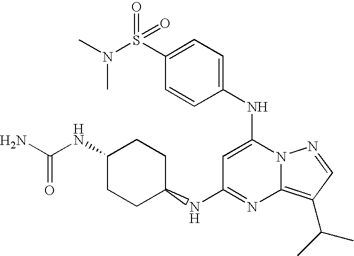 | 141 | 0.142 | 13 | 9 | 515 (M + H, 100%) | 2.18 |
| 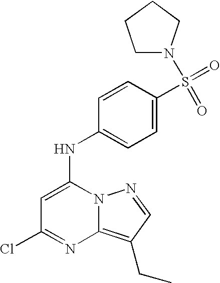 | 142 | 0.066 | ND | >200 | 406 and 408 (each M + H, 100 and 35%) | 2.72 |
| 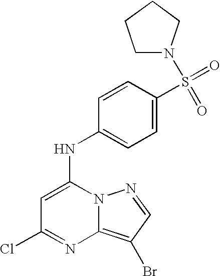 | 143 | ND | ND | ND | 456, 458 and 460 (each M + H, 75, 100 and 25%) | 2.65 |
| 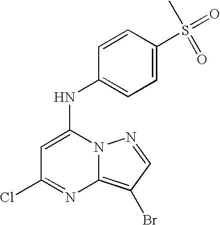 | 144 | 0.23 | ND | >50 | 401, 403 and 405 (each M + H, 75, 100 and 25%) | 2.32 |
| 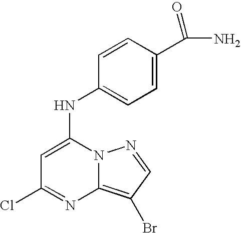 | 145 | 0.08 | 11 | >50 | 366, 368 and 370 (each M + H, 75, 100 and 25%) | 2.13 |
| 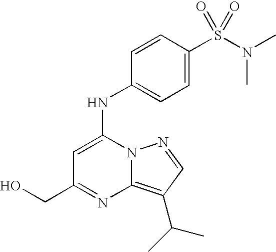 | 146 | 0.546 | ND | ND | 390 (M + H, 100%) | 2.31 |
| 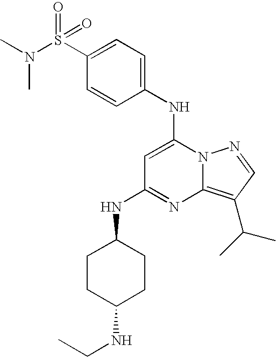 | 147 | 0.098 | 4 | 10 | 500 (M + H, 100%) | 1.94 |
| 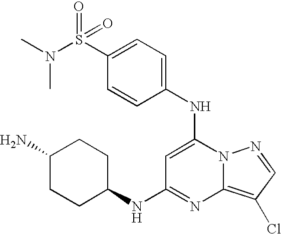 | 148 | 0.004 | 2 | 1.545 | 464 and 466 (each M + H, 100 and 35%) | 1.86 |
| 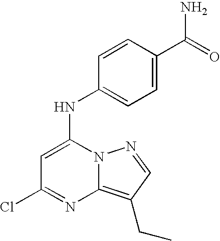 | 149 | 0.14 | 10 | 57 | 316 and 318 (each M + H, 100 and 35%) | 2.21 |
| 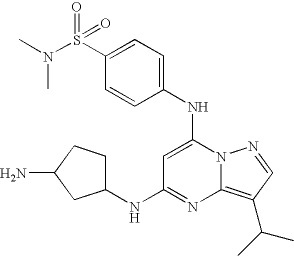 | 150 | 0.478 | 2 | 13 | 458 (M + H, 100%) | 1.92 |
| 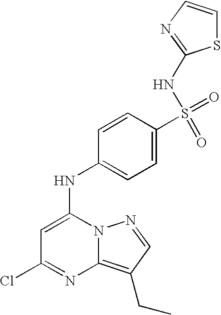 | 151 | 0.11 | ND | 38 | 435 and 437 (each M + H, 100 and 35%) | 2.38 |
| 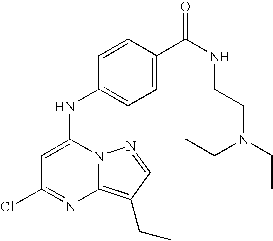 | 152 | 0.79 | 32 | ND | 415 and 417 (each M + H, 100 and 35%) | 1.92 |
| 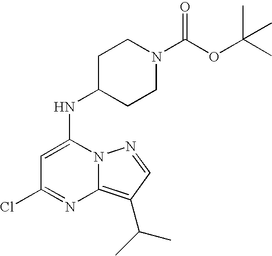 | 153 | 3.437 | ND | ND | 394 and 396 (each M + H, 100 and 35%) | 2.91 |
| 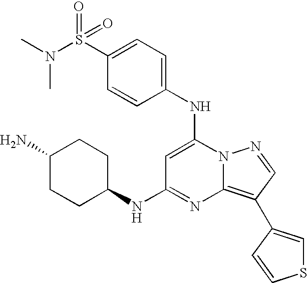 | 154 | 1.421 | 0.63 | >200 | 512 (M + H, 100%) | 2.04 |
| 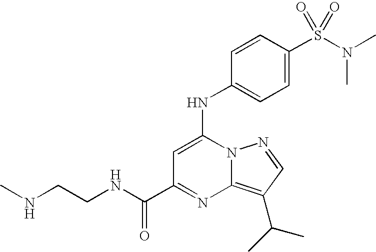 | 155 | 0.142 | 53 | ND | 460 (M + H, 100%) | 1.93 |
| 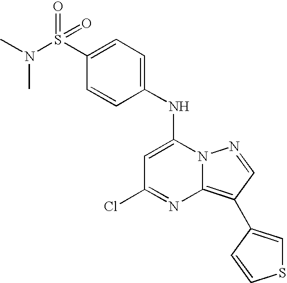 | 156 | 24.866 | >200 | 10 | 434 and 436 (each M + H, 100 and 35%) | 2.78 |
| 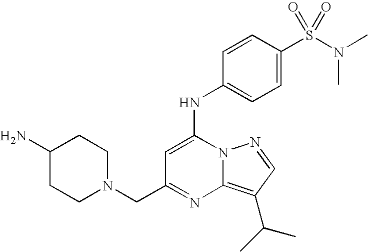 | 157 | 0.037 | 8 | 72 | 472 (M + H, 100%) | 1.66 |
| 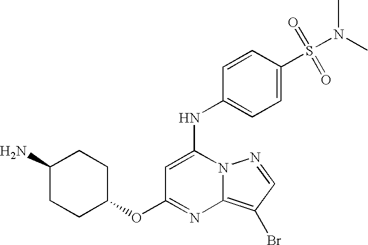 | 158 | 0.005 | ND | ND | 509 and 511 M + H, 100%), 531 and 533 M + Na, 100%) | 1.94 |
| 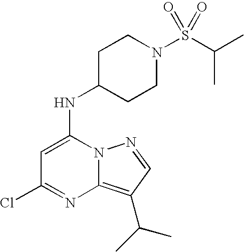 | 159 | 1.172 | ND | ND | 400 and 402 (each M + H, 100 and 35%) | 2.70 |
| 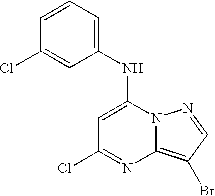 | 160 | 0.108 | ND | ND | 357, 359 and 361 (each M + H, 75, 100 and 25%) | 2.79 |
| 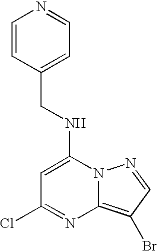 | 161 | 0.230 | ND | ND | 338, 340 and 342 (each M + H, 75, 100 and 25%) | 1.92 |
| 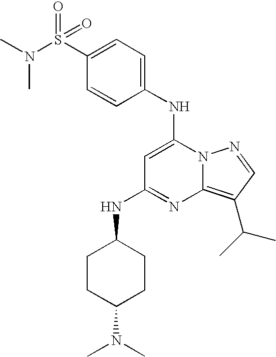 | 162 | 0.815 | 12 | 5 | 500 (M + H, 100%) | 2.01 |
| 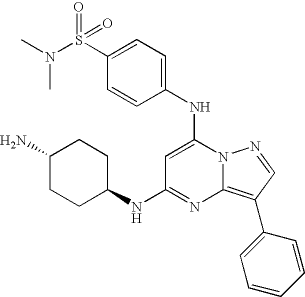 | 163 | 10 | 1.53 | 6 | 506 (M + H, 100%) | 2.08 |
| 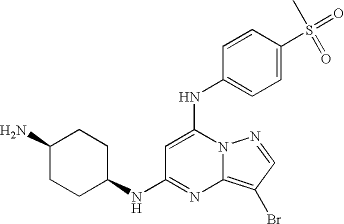 | 164 | 0.222 | 0.25 | 4.70 | 479 and 481 (each M + H, 100%) | 1.83 |
| 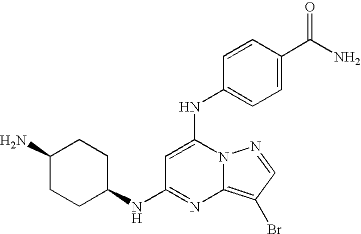 | 165 | 0.026 | 0.62 | 1.20 | 444 and 446 (each M + H, 100%) | 1.70 |
| 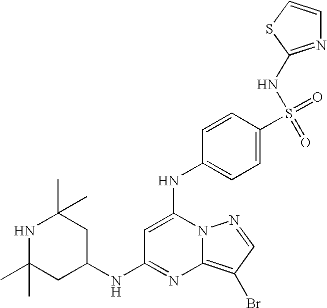 | 166 | 16.859 | 17 | 2.15 | 605 and 607 (each M + H, 100%) | 1.91 |
| 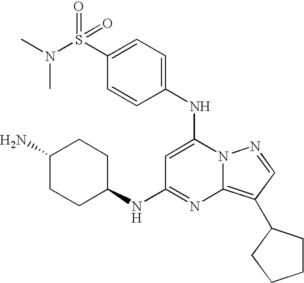 | 167 | 0.004 | 0.72 | 168.312 | 498 (M + H, 100%) | 2.02 |
| 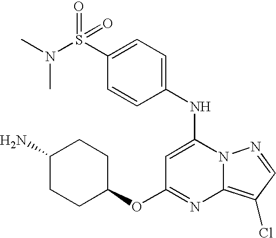 | 168 | 0.014 | 5 | 15.190 | 487 and 489 (each M + Na, 100 and 35%) | 1.90 |
| 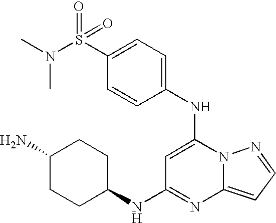 | 169 | 0.098 | ND | 3.359 | 430 (M + H, 100%) | 1.63 |
| 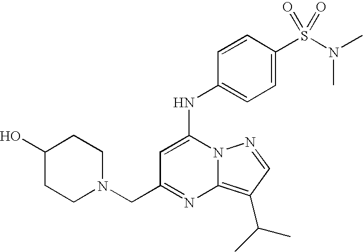 | 170 | 1.804 | ND | >200 | 473 (M + H, 100%) | 1.87 |
| 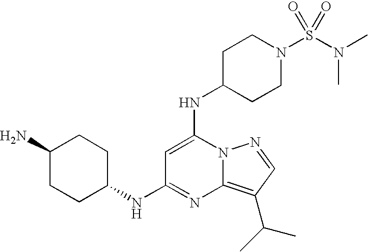 | 171 | 11.861 | ND | >200 | 478 (M + H, 100%) | 1.73 |
| 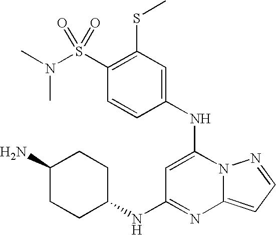 | 172 | >50 | ND | 72.4 | 465 (M + H, 100%) | 1.74 |
| 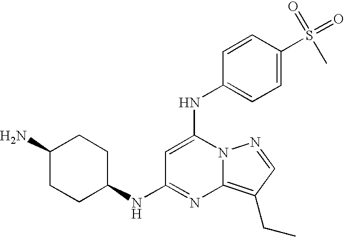 | 173 | 2.173 | 4 | 15.20 | 429 (M + H, 100%) | 1.75 |
| 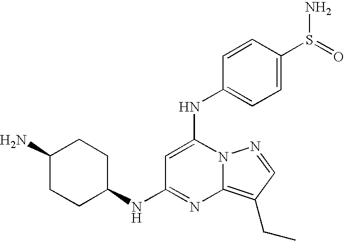 | 174 | 0.35 | ND | 11.28 | 394 (M + H, 100%) | 1.62 |
| 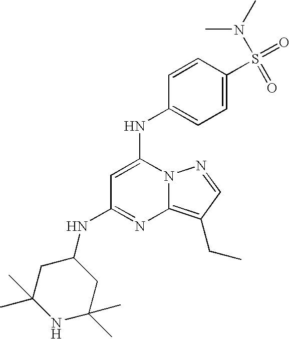 | 175 | 22.153 | ND | 12.88 | 500 (M + H, 100%) | 2.01 |
| 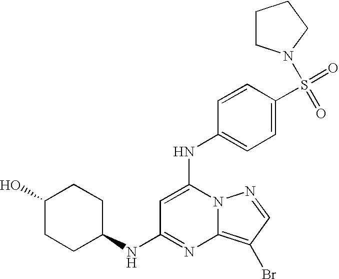 | 176 | 0.183 | ND | 3.25 | 535 and 537 (M + H, 100%) | 242 |
| | | CDK2 | Chk1 | PDK1 | | RT |
| Structure | Example | IC₅₀(μM) | IC₅₀(μM) | IC₅₀(μM) | m/z | (min) |
| 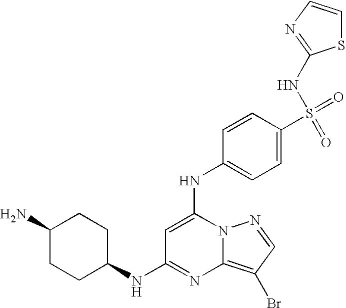 | 177 | ND | ND | 0.66 | 563 and 565 (M + H, 100%) | 1.83 |
| 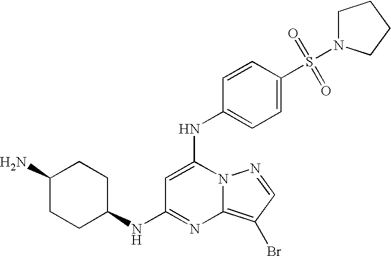 | 178 | 0.15 | 0.07 | 2.58 | 534 and 536 (M + H, 100%) | 2.00 |
| 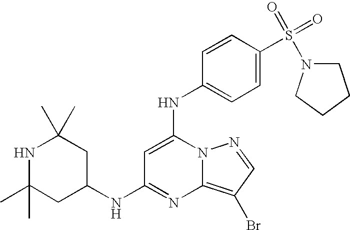 | 179 | 24 | ND | 4.54 | 576 and 578 (M + H, 100%) | 2.10 |
| 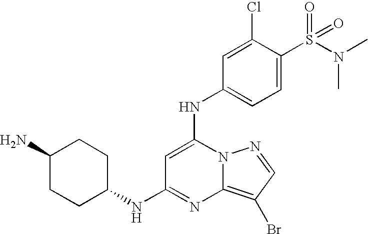 | 180 | 0.002 | 0.83 | 2 | 542, 544 and 546 (each M + H, 75, 100 and 25%) | 1.97 |
| 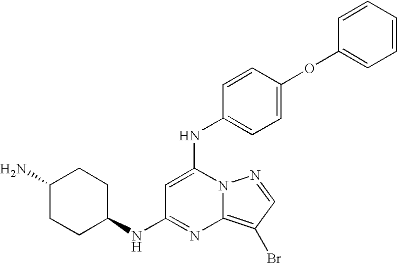 | 181 | 0.101 | 1 | 109 | 493 and 495 (M + H, 100%) | 2.20 |
| 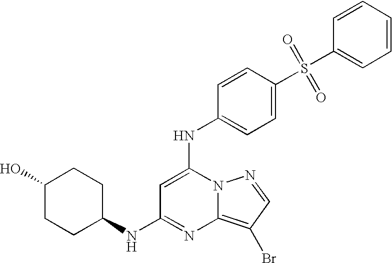 | 182 | 0.248 | ND | 3 | 542 and 544 (M + H, 100%) | 2.48 |
| 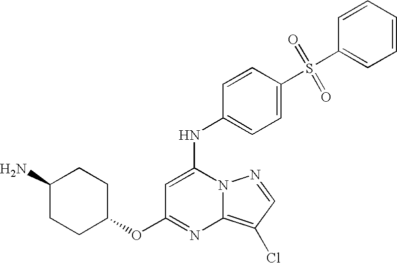 | 183 | 0.015 | ND | ND | 497 and 499 (each M + H, 100 and 35%) | 1.97 |
| 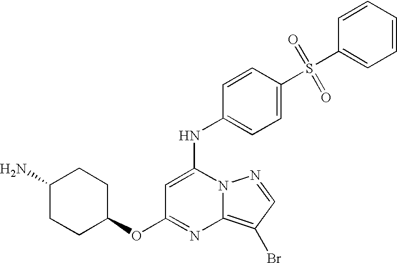 | 184 | 0.015 | ND | ND | 564 and 566 (M + H, 100%) | 2.06 |
| 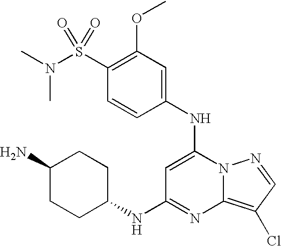 | 185 | 0.27 | 9 | 18 | 538 and 540 (M + H, 100%) | 1.91 |
| 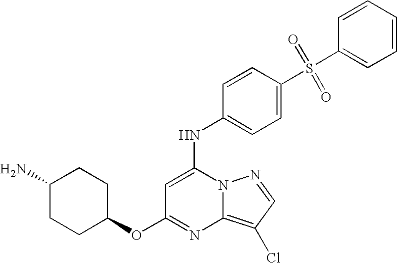 | 186 | 0.045 | 2 | 2 | 520 and 522 (each M + H, 100 and 35%) | 2.05 |
| 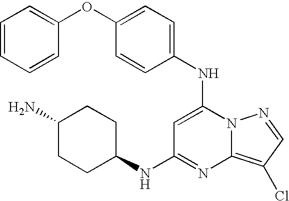 | 187 | 0.054 | 2 | 2 | 449 and 451 (each M + H, 100 and 35%) | 2.18 |
| 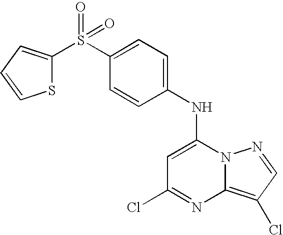 | 188 | 0.089 | ND | ND | 425, 427 and 429 (each M + H, 100, 67 and 11%) | 2.66 |
| 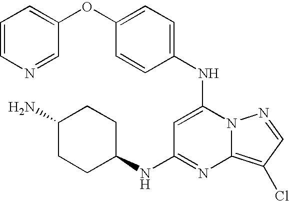 | 189 | 0.028 | ND | ND | 450 (M + H, 100%) | 1.81 |
| 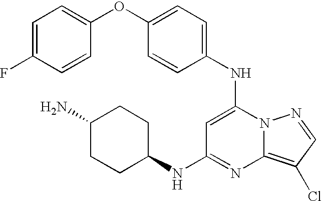 | 190 | 0.046 | ND | ND | 467 (M + H, 100%) | 2.19 |
| 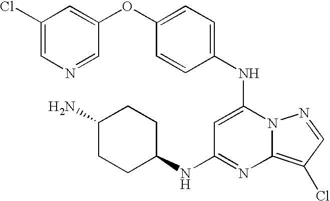 | 191 | 0.018 | ND | ND | 484, and 486 (each M + H, 100 and 67%) | 2.03 |
| 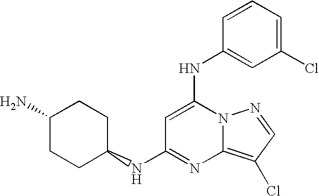 | 192 | 0.009 | 2.14 | 3.24 | 391, 393 and 395 (each M + H, 100, 67 and 11%) | 2.00 |
| 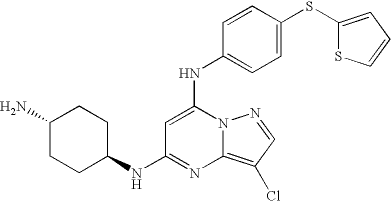 | 193 | 0.047 | 1.47 | 2.88 | 471 and 473 (each M + H, 100 and 35%) | 2.24 |
| 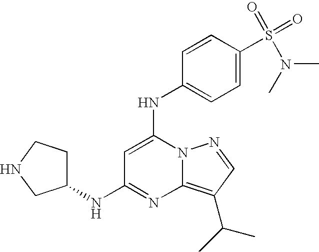 | 194 | 0.073 | 0.21 | 4.42 | 444 (M + H, 100%) | 2.03 |
| 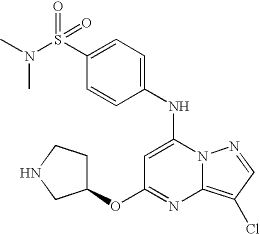 | 195 | 0.24 | 7.79 | ND | 437 and 439 (each M + H, 100 and 35%) | 1.81 |
| 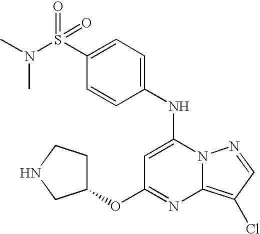 | 196 | 0.36 | 4.19 | ND | 437 and 439 (each M + H, 100 and 35%) | 1.87 |
| 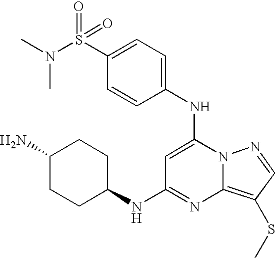 | 197 | 0.055 | ND | ND | 476 (M + H, 100%) | 1.84 |
| 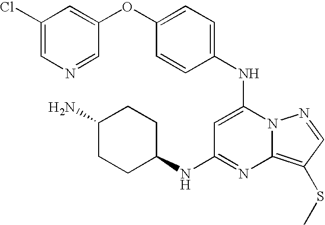 | 198 | 0.018 | 2.08 | 4.48 | 496 and 498 (each M + H, 100 and 40%) | 2.02 |
| 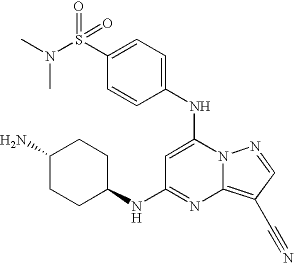 | 199 | 0.007 | 0.33 | 1.35 | 455 (M + H, 100%) | 1.83 |
| 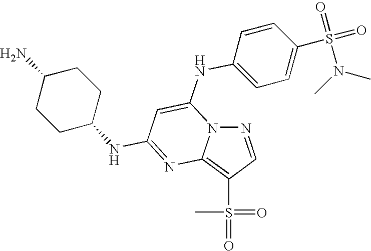 | 200 | 11.17 | ND | ND | | |

In the above TABLE “ND“means the compound was not tested in that assay.

Assay Conditions:
A. Enzyme Inhibition Assays
CDK2

Assays for the cyclin dependent kinase activity were carried out by monitoring the phosphorylation of a synthetic peptide, HATTPKKKRK. The assay mixture containing the inhibitor and CDK-2 enzyme, complexed with cyclin A (0.4 U/ml) was mixed together in a microtiter plate in a final volume of 50 μl and incubated for 40 min at 30° C. The assay mixture contained 0.1 mM unlabeled ATP, 0.01 μCi/μl ³³P-γ-ATP, 0.03 mM peptide, 0.1 mg/ml BSA, 7.5 mM magnesium acetate, 50 mM HEPES-NaOH, pH 7.5. The reaction was stopped by adding 50 μl of 50 mM phosphoric acid. 90 μl of the mixture were transferred to a pre-wetted 96-well Multiscreen MAPHNOB filtration plate (Millipore) and filtered on a vacuum manifold. The filter plate was washed with 3 successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer)

HEPES is N-[2-Hydroxyethyl]piperazine-N′-[2-ethanesulfonic acid] BSA is bovine serum albumin.

PDK1

Assays for the PDK dependent kinase activity were carried out by monitoring the phosphorylation of a synthetic peptide, KTFCGTPEYLAPEVRREPRILSEEEQEMFRDFDYIADWC. The assay mixture containing the inhibitor and PDK1 enzyme was mixed together in a microtiter plate in a final volume of 50 μl and incubated for 60 min at 30° C. The assay mixture contained 0.01 mM unlabeled ATP, 0.01 μCi/μl ³³P-γ-ATP, 0.075 mM peptide, 0.1 mg/ml BSA, 7.5 mM magnesium acetate, 0.05M Tris.HCl, pH 7.5, 0.5% 2-mercaptoethanol. The reaction was stopped by adding 50 μl of 50 mM phosphoric acid. 90 μl of the mixture were transferred to a pre-wetted 96-well Multiscreen MAPHNOB filtration plate (Millipore) and filtered on a vacuum manifold. The filter plate was washed with 3 successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer)

CHK1:

Assays for the Chk1 kinase activity were carried out by monitoring the phosphorylation of a synthetic peptide Chktide with the amino acid sequence, KKKVSRSGLYRSPSMPENLNRPR. The assay mixture containing the inhibitor and Chk1 enzyme was mixed together in a microtiter plate in a final volume of 50 μl and incubated for 40 minutes at 30° C.

The assay mixture contained 0.01 mM unlabeled ATP, 0.51 μCi ³³P-γ-ATP, 30 μM Chktide, 0.1 mg/ml BSA, 50 mM Hepes-NaOH pH 7.5 and 11 nM GST-Chk1 enzyme. The reaction was stopped by adding 50 μl of 50 mM phosphoric acid. 90 μl of the mixture was transferred to a pre-wetted 96-well Multiscreen MAPHNOB filtration plate (Millipore) and filtered on a vacuum manifold. The filter plate was washed with 3 successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer)

B. Cell Growth Inhibition Assay:

Assessment of Cytotoxicity by Sulforhodamine B (SRB) Assay: Calculation of 50% Inhibitory Concentration (IC₅₀).

Day 1

1) Determine cell number by haemocytometer.
2) Using an 8 channel multipipettor, add 160 μl of the cell suspension (3600 cells/well or 2×10⁴ cells/ml) to each well of a 96-well microtitre plate.
3) Incubate overnight at 37° C. in a CO₂ incubator.
   Day 2
4) Stock solutions of drugs are prepared, and serial dilutions of each drug are performed in medium to give final concentrations in wells.
5) Using a multipipettor, 40 μl of drug (at 5× final concentration) is added to quadruplicate wells.
6) Control wells are at either side of the 96 well plates, where 40 μl of medium is added.
7) Incubate plates in CO₂ incubator for 4 days
   Day 6
8) Tip off medium into sink and immerse plate slowly into 10% ice cold trichloroacetic acid (TCA). Leave for about 30 mins on ice.
9) Wash plates three times in tap water by immersing the plates into baths of tap water and tipping it off.
10) Dry in incubator.
11) Add 100 μl of 0.4% SRB in 1% acetic acid to each well (except the last row right hand)of the 96 well plate, this is the 0% control, ie no drug, no stain. The first row will be the 100% control with no drug, but with stain). Leave for 5 mins.
12) Wash off unbound SRB stain with four washes of 1% acetic acid.
13) Dry plates in incubator.
14) Solubilise SRB using 100 μl of 10 mM Tris base and put plates on plate shaker for 5 mins.
15) Determine absorbance at 540 nm using a plate reader. Calculate mean absorbance for quadruplicate wells and express as a percentage of value for control, untreated wells.

Plot % absorbance values versus log drug concentration and determine the IC₅₀.

By way of illustration, the results obtained for some of the above example compounds are given in the following Table:

| Example | GI₅₀ (μM) |
|---|---|
| 147 | 1 |
| 168 | 0.21 |
| 180 | 0.33 |
| 181 | 0.09 |
| 191 | 0.04 |
| 192 | 0.2 |
| 194 | 0.59 |
| 196 | 1 |
| 197 | 0.58 |
| 198 | 0.31 |

1. A compound of formula (I) or a salt, N-oxide, hydrate or solvate thereof, for inhibition of kinase activity: 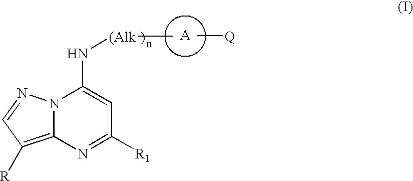
wherein
Ring A is an optionally substituted carbocyclic or heterocyclic radical,
Alk represents an optionally substituted divalent C₁-C₆ alkylene radical;
n is 0 or 1;
Q represents a radical of formula -(Alk¹)ₚ-(X)ᵣ-(Alk²)ₛ-Z wherein in any compatible combination
Z is hydrogen or an optionally substituted carbocyclic or heterocyclic ring,
Alk¹ and Alk² are optionally substituted divalent C₁-C₆ alkylene radicals which may contain a —O—, —S—or —NR^{A}-link, wherein R^{A} is hydrogen or C₁-C₆ alkyl,
X represents —O—, —S—, —(C═O)—, —(C═S)—, —SO₂—, —SO—, —C(═O)O—, —OC(═O)—, —C(═O)NR^{A}—, .NR^{A}C(═O)—, —C(═S)NR^{A}—, —NR^{A}C(═S)—, —SO₂NR^{A}., —NR^{A}SO₂—, —OC(═O)NR^{A}—, —NR^{A}C(═O)O—, or —NR^{A}— wherein R^{A} is hydrogen or C₁-C₆ alkyl, and
p, r and s are independently 0 or 1,
R₁ represents a radical -(Alk³)ₐ-(Y)_{b}-(Alk⁴)_{d}-B wherein a, b and d are independently 0 or 1,
Alk³ and Alk⁴ are optionally substituted divalent C₁-C₃ alkylene radicals,
Y represents a monocyclic divalent carbocyclic or heterocyclic radical having from 5 to 8 ring atoms, —O—, —S—, or —NR^{A}— wherein R^{A} is hydrogen or C₁-C₆ alkyl,
B represents hydrogen or halo, or an optionally substituted monocyclic carbocyclic or heterocyclic ring having from 5 to 8 ring atoms, or in the case where Y is —NR^{A}— and b is 1, then R^{A} and the radical-(Alk⁴)_{d}-B taken together with the nitrogen to which they are attached may form an optionally substituted heterocyclic ring,
R represents hydrogen, halo, C₁-C₆ alkyl, C₁-C₆ alkoxy, C₁-C₆ alkylthio, phenyl, benzyl, cycloalkyl with 3 to 6 ring atoms, or a monocyclic heterocyclic group having 5 or 6 ring atoms.

2. The compound as claimed in claim 1 wherein ring A is an optionally substituted monocyclic aryl or heteroaryl radical.

3. The compound as claimed in claim 2 wherein ring A is phenyl, naphthyl, 2-, 3- and 4-pyridyl, 5-pyrimidinyl, 2- and 3-thienyl, 2- and 3-furyl, piperazinyl, pyrrolidinyl, or thiazolinyl.

4. The compound as claimed in claim 1 wherein ring A is phenyl.

5. The compound as claimed in claim 1 wherein ring A is unsubstituted or substituted by methyl, ethyl, methylenedioxy, ethylenedioxy, methoxy, ethoxy, methylthio, ethylthio, hydroxy, hydroxymethyl, hydroxyethyl, mercapto, mercaptomethyl, mercaptoethyl, amino, mono- or di-methylamino, mono- or di-ethylamino, fluoro, chloro, bromo, cyano, N-morpholino, N-piperidinyl, or N-piperazinyl, the latter being optionally C₁-C₆ alkyl- or benzyl-substituted on the free ring nitrogen, dimethylaminosulfonyl, phenylsulfonyl or phenoxy.

6. The compound as claimed in claim 1 wherein Q is hydrogen and the ring A is 4-(dimethylaminosulfonyl)-phenyl, 4-(phenylsulfonyl)-phenyl, 4-(phenoxy)-phenyl, 3-chloro-4-(dimethylaminosulfonyl)-phenyl, 3-chloro-4(phenylsulfonyl)-phenyl, 3-chloro-4-(phenoxy)-phenyl, 3-methoxy-4(dimethylaminosulfonyl)-phenyl, 3-methoxy-4-(phenylsulfonyl)-phenyl, or 3-methoxy-4-(phenoxy)-phenyl.

7. The compound as claimed in claim 1 wherein n is 1 and Alk is CH₂—, —CH₂CH₂—, —CH₂CH(CH₃)—, —CH₂CH₂CH₂—, —CH═CH—, —CH₂CH═CH—, —CH₂CH═CHCH₂—, —CH═CHCH═CH—, —C═C—, —CH₂C═C—, or —CH₂C═CCH₂—.

8. The compound as claimed in claim 1 wherein n is 1 and Alk is —CH₂—.

9. The compound as claimed in claim 1 wherein n is 0.

10. The compound as claimed in claim 1 wherein each of p, r and s is 0, and Z is hydrogen.

11. The compound as claimed in claim 1 wherein p, r and s are each 0, and Z is an optionally substituted monocyclic carbocyclic or heterocyclic ring.

12. The compound as claimed in claim 11 wherein Z is an optionally substituted phenyl, cyclopentyl, cyclohexyl, pyridyl, morpholino, piperidinyl, or piperazyl ring.

13. The compound as claimed in claim 1 wherein one or more of p, r and s is 1, and Z is hydrogen or an optionally substituted monocyclic carbocyclic or heterocyclic ring.

14. The compound as claimed in claim 13 wherein p, or both are each 1 and r is 0.

15. The compound as claimed in claim 13 wherein each of p, r, and s is 1.

16. The compound as claimed in claim 13 wherein p and s are each 0 and r is 1.

17. The compound as claimed in claim 16 wherein X is —SO₂—, —O—, a sulfonamide radical —NR^{A}SO₂— or a carboxamide radical —NR^{A}C(═O)— with the N atom linked to the ring A.

18. The compound as claimed in claim 13 wherein p is 0, r is 1, s is 1 or 0, and X is a sulfonamide radical —NR^{A}SO₂— or a carboxamide radical —NR^{A}C(═O)— with the N atom linked to the ring A.

19. The compound as claimed in claim 17 wherein R^{A} is hydrogen or methyl.

20. The compound as claimed in claim 18 wherein s is 1 and Z is hydrogen.

21. The compound as claimed in claim 18 or wherein s is 0 and Z is an optionally substituted monocyclic carbocyclic or heterocyclic ring.

22. The compound as claimed in claim 21 wherein Z is optionally substituted phenyl.

23. The compound as claimed in claim 1 wherein in the radical R₁ a, b and d are all 0.

24. The compound as claimed in claim 1 wherein in the radical R₁ a and d are each 0 and b is 1.

25. The compound as claimed in claim 1 wherein in the radical R₁ b is 0 and at least one of a and d is 1.

26. The compound as claimed in claim 23 wherein in the radical R₁, B is an optionally substituted monocyclic carbocyclic or heterocyclic ring.

27. The compound as claimed in claim 26 wherein B is an optionally substituted cyclopentyl, cyclohexyl, phenyl, 2-, 3-, or 4-pyridyl, 2-, or 3-thienyl, 2-, or 3-furanyl, pyrrolyl, pyranyl, or piperidinyl ring.

28. The compound as claimed in claim 27 wherein optional substituents are selected from methyl, ethyl, methoxy, ethoxy, methylenedioxy, ethylenedioxy, methylthio, ethylthio, hydroxy, hydroxymethyl, hydroxyethyl, mercapto, mercaptomethyl, mercaptoethyl, amino, mono- and di-methylamino, mono- and di-ethylamino, fluoro, chloro, bromo, cyano, N-morpholino, N-piperidinyl, N-piperazinyl.

29. The compound as claimed in claim 1 wherein R₁ is optionally substituted cyclohexyloxy; cyclohexylamino; cyclohexylmethyl, or piperidin-1ylmethyl.

30. The compound as claimed in claim 1 wherein R₁ is 4-aminocyclohexyloxy; 4-aminocyclohexylamino; 4-hydroxycyclohexylamino, 4-aminocyclohexylmethyl, or 4-aminopiperidin-1-ylmethyl.

31. The compound as claimed in claim 1 wherein R is hydrogen, chloro, bromo methyl, ethyl, n-propyl, iso-propyl, n-, sec- or tert-butyl, methoxy, methylthio, ethoxy, ethylthio, or a phenyl, benzyl, cyclopropyl, cyclopentyl, cyclohexyl, 2-, 3-, or 4-pyridyl, phenyl, pyridyl, morpholino, piperidinyl, or piperazyl ring.

32. The compound as claimed in claim 1 wherein R is chloro, bromo, cyclopentyl, cyclopropyl or isopropyl.

33. The compound as claimed in claim 1 wherein in the compound of formula (I) n is 0, ring A is optionally substituted phenyl, Q is dimethylaminosulfonyl, phenylsulfonyl or phenoxy; R¹ is 4-aminocyclohexyloxy, 4-aminocyclohexylamino, 4-hydroxycyclohexylamino, 4-aminocyclohexylmethyl, or 4-aminopiperidin-1-ylmethyl, and R is chloro, bromo, cyclopentyl, cyclopropyl or isopropyl.

34. A method of treatment of diseases or conditions mediated by excessive or inappropriate kinase activity in mammals, comprising administering to the mammal an amount of a compound of formula (I) as defined in claim 1, or a salt, hydrate or solvate thereof, effective to inhibit said kinase activity.

35. (canceled)

36. The method as claimed in claim 34, wherein the kinase activity is CDK2 activity, PDK1 activity, CHK1 activity, or combinations thereof.

37. The method of treatment as claimed in claim 34, wherein the kinase activity is associated with cancer, psoriasis or restenosis.

38. A pharmaceutical composition comprising a compound of formula (I) as defined in claim 1, or a salt, N-oxide, hydrate or solvate thereof, together with a pharmaceutically acceptable carrier.

39. A compound of formula (I), or a salt, N-oxide, hydrate or solvate thereof, 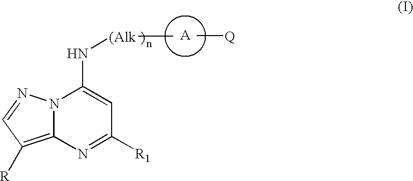
wherein n is 0, ring A is optionally substituted phenyl, Q is dimethylaminosulfonyl, phenylsulfonyl or phenoxy, R¹ is 4-aminocyclohexyloxy; 4-aminocyclohexylamino; 4-hydroxyyclohexylamino; 4-aminocyclohexylmethyl, or 4-aminopiperidin-1-ylmethyl, and R is chloro, bromo, cyclopentyl, cyclopropyl or isopropyl.

40. A pharmaceutical composition as claimed in claim 39 together with a pharmaceutically acceptable carrier.
